SEQUENCE LISTING (42 sequences)

SEQ ID NO: 1 - protein, 20 aa, synthetic construct
  REGION 1..20: note = Polypeptide sequence of linker used in constructs ID3A and ID55F
GGGGSGGGGSGGGGSGGGGS

SEQ ID NO: 2 - protein, 11 aa, synthetic construct
  REGION 1..11: note = Polypeptide sequence of linker used in constructs ID25A and ID57F
GGGGSKGGGGS

SEQ ID NO: 3 - protein, 13 aa, synthetic construct
  REGION 1..13: note = Polypeptide sequence of linker used in constructs ID26A and ID58F
GGGGSDKDGGGGS

SEQ ID NO: 4 - protein, 15 aa, synthetic construct
  REGION 1..15: note = Polypeptide sequence of linker used in constructs ID27A and ID59F
GGGGSDDDDKGGGGS

SEQ ID NO: 5 - protein, 12 aa, synthetic construct
  REGION 1..12: note = Polypeptide sequence of linker used in constructs ID28A and ID60F
KGGGGSGGGGSK

SEQ ID NO: 6 - protein, 13 aa, synthetic construct
  REGION 1..13: note = Polypeptide sequence of linker used in construct ID56F
RGGGGSRGGGGSR

SEQ ID NO: 7 - protein, 242 aa, synthetic construct
  REGION 1..242: note = Polypeptide sequence of ID3A construct
DVQLQESGGGLVQPGGSLRLSCVISGMDFSHKPAGWFRQAPGKEREFVASITTRASTHYADSVKGRFTISRDNAKNTVYLEMNSLKPEDTAVYSCNSEYYWGQGTQVTVSSGGGGSGGGGSGGGGSGGGGSDVQLQESGGGLVQPGGSLRLSCVISGMDFSHKPAGWFRQAPGKEREFVASITTRASTHYADSVKGRFTISRDNAKNTVYLEMNSLKPEDTAVYSCNSEYYWGQGTQVTVSS

SEQ ID NO: 8 - protein, 233 aa, synthetic construct
  REGION 1..233: note = Polypeptide sequence of ID25A construct
DVQLQESGGGLVQPGGSLRLSCVISGMDFSHKPAGWFRQAPGKEREFVASITTRASTHYADSVKGRFTISRDNAKNTVYLEMNSLKPEDTAVYSCNSEYYWGQGTQVTVSSGGGGSKGGGGSDVQLQESGGGLVQPGGSLRLSCVISGMDFSHKPAGWFRQAPGKEREFVASITTRASTHYADSVKGRFTISRDNAKNTVYLEMNSLKPEDTAVYSCNSEYYWGQGTQVTVSS

SEQ ID NO: 9 - protein, 235 aa, synthetic construct
  REGION 1..235: note = Polypeptide sequence of ID26A construct
DVQLQESGGGLVQPGGSLRLSCVISGMDFSHKPAGWFRQAPGKEREFVASITTRASTHYADSVKGRFTISRDNAKNTVYLEMNSLKPEDTAVYSCNSEYYWGQGTQVTVSSGGGGSDKDGGGGSDVQLQESGGGLVQPGGSLRLSCVISGMDFSHKPAGWFRQAPGKEREFVASITTRASTHYADSVKGRFTISRDNAKNTVYLEMNSLKPEDTAVYSCNSEYYWGQGTQVTVSS

SEQ ID NO: 10 - protein, 237 aa, synthetic construct
  REGION 1..237: note = Polypeptide sequence of ID27A construct
DVQLQESGGGLVQPGGSLRLSCVISGMDFSHKPAGWFRQAPGKEREFVASITTRASTHYADSVKGRFTISRDNAKNTVYLEMNSLKPEDTAVYSCNSEYYWGQGTQVTVSSGGGGSDDDDKGGGGSDVQLQESGGGLVQPGGSLRLSCVISGMDFSHKPAGWFRQAPGKEREFVASITTRASTHYADSVKGRFTISRDNAKNTVYLEMNSLKPEDTAVYSCNSEYYWGQGTQVTVSS

SEQ ID NO: 11 - protein, 234 aa, synthetic construct
  REGION 1..234: note = Polypeptide sequence of ID28A construct
DVQLQESGGGLVQPGGSLRLSCVISGMDFSHKPAGWFRQAPGKEREFVASITTRASTHYADSVKGRFTISRDNAKNTVYLEMNSLKPEDTAVYSCNSEYYWGQGTQVTVSSKGGGGSGGGGSKDVQLQESGGGLVQPGGSLRLSCVISGMDFSHKPAGWFRQAPGKEREFVASITTRASTHYADSVKGRFTISRDNAKNTVYLEMNSLKPEDTAVYSCNSEYYWGQGTQVTVSS

SEQ ID NO: 12 - protein, 268 aa, synthetic construct
  REGION 1..268: note = Polypeptide sequence of ID55F construct
EVQLVESGGGLVESGASLRLSCVTSGHIFKLYGMGWFRQAPGKDREFVAAVTWNGPSTEYADSVKGRFTISKDNDRNTLYLQMNSLKPEDTAVYYCARSSIMGIYTTPDRYEYWGQGTQVTVSSGGGGSGGGGSGGGGSGGGGSEVQLVESGGGLVESGASLRLSCVTSGHIFKLYGMGWFRQAPGKDREFVAAVTWNGPSTEYADSVKGRFTISKDNDRNTLYLQMNSLKPEDTAVYYCARSSIMGIYTTPDRYEYWGQGTQVTVSS

SEQ ID NO: 13 - protein, 259 aa, synthetic construct
  REGION 1..259: note = Polypeptide sequence of ID57F construct
EVQLVESGGGLVESGASLRLSCVTSGHIFKLYGMGWFRQAPGKDREFVAAVTWNGPSTEYADSVKGRFTISKDNDRNTLYLQMNSLKPEDTAVYYCARSSIMGIYTTPDRYEYWGQGTQVTVSSGGGGSKGGGGSEVQLVESGGGLVESGASLRLSCVTSGHIFKLYGMGWFRQAPGKDREFVAAVTWNGPSTEYADSVKGRFTISKDNDRNTLYLQMNSLKPEDTAVYYCARSSIMGIYTTPDRYEYWGQGTQVTVSS

SEQ ID NO: 14 - protein, 261 aa, synthetic construct
  REGION 1..261: note = Polypeptide sequence of ID58F construct
EVQLVESGGGLVESGASLRLSCVTSGHIFKLYGMGWFRQAPGKDREFVAAVTWNGPSTEYADSVKGRFTISKDNDRNTLYLQMNSLKPEDTAVYYCARSSIMGIYTTPDRYEYWGQGTQVTVSSGGGGSDKDGGGGSEVQLVESGGGLVESGASLRLSCVTSGHIFKLYGMGWFRQAPGKDREFVAAVTWNGPSTEYADSVKGRFTISKDNDRNTLYLQMNSLKPEDTAVYYCARSSIMGIYTTPDRYEYWGQGTQVTVSS

SEQ ID NO: 15 - protein, 263 aa, synthetic construct
  REGION 1..263: note = Polypeptide sequence of ID59F construct
EVQLVESGGGLVESGASLRLSCVTSGHIFKLYGMGWFRQAPGKDREFVAAVTWNGPSTEYADSVKGRFTISKDNDRNTLYLQMNSLKPEDTAVYYCARSSIMGIYTTPDRYEYWGQGTQVTVSSGGGGSDDDDKGGGGSEVQLVESGGGLVESGASLRLSCVTSGHIFKLYGMGWFRQAPGKDREFVAAVTWNGPSTEYADSVKGRFTISKDNDRNTLYLQMNSLKPEDTAVYYCARSSIMGIYTTPDRYEYWGQGTQVTVSS

SEQ ID NO: 16 - protein, 260 aa, synthetic construct
  REGION 1..260: note = Polypeptide sequence of ID60F construct
EVQLVESGGGLVESGASLRLSCVTSGHIFKLYGMGWFRQAPGKDREFVAAVTWNGPSTEYADSVKGRFTISKDNDRNTLYLQMNSLKPEDTAVYYCARSSIMGIYTTPDRYEYWGQGTQVTVSSKGGGGSGGGGSKEVQLVESGGGLVESGASLRLSCVTSGHIFKLYGMGWFRQAPGKDREFVAAVTWNGPSTEYADSVKGRFTISKDNDRNTLYLQMNSLKPEDTAVYYCARSSIMGIYTTPDRYEYWGQGTQVTVSS

SEQ ID NO: 17 - protein, 261 aa, synthetic construct
  REGION 1..261: note = Polypeptide sequence of ID56F construct
EVQLVESGGGLVESGASLRLSCVTSGHIFKLYGMGWFRQAPGKDREFVAAVTWNGPSTEYADSVKGRFTISKDNDRNTLYLQMNSLKPEDTAVYYCARSSIMGIYTTPDRYEYWGQGTQVTVSSRGGGGSRGGGGSREVQLVESGGGLVESGASLRLSCVTSGHIFKLYGMGWFRQAPGKDREFVAAVTWNGPSTEYADSVKGRFTISKDNDRNTLYLQMNSLKPEDTAVYYCARSSIMGIYTTPDRYEYWGQGTQVTVSS

SEQ ID NO: 18 - protein, 111 aa, synthetic construct
  REGION 1..111: note = Polypeptide sequence of ID1A ICVD
DVQLQESGGGLVQPGGSLRLSCVISGMDFSHKPAGWFRQAPGKEREFVASITTRASTHYADSVKGRFTISRDNAKNTVYLEMNSLKPEDTAVYSCNSEYYWGQGTQVTVSS

SEQ ID NO: 19 - protein, 124 aa, synthetic construct
  REGION 1..124: note = Polypeptide sequence of ID5F ICVD
DVQLQESGGGLVESGASLRLSCVTSGHIFKLYGMGWFRQAPGKDREFVAAVTWNGPSTEYADSVKGRFTISKDNDRNTLYLQMNSLKPEDTAVYYCARSSIMGIYTTPDRYEYWGQGTQVTVSS

SEQ ID NO: 20 - DNA, 732 bp, synthetic construct
  misc_feature 1..732: note = Polynucleotide sequence encoding ID3A construct
  source 1..732: mol_type = other DNA
gatgtgcagctgcaggagtctgggggaggcttggtgcagcctggggggtctctgagactctcctgtgtaatctctggaatggacttcagtcacaaacccgcgggctggttccgccaggctccaggaaaagagcgcgagttcgtcgcttcgattacgactcgtgctagcacgcactatgcagactccgtgaagggccgattcaccatctccagagacaacgccaagaacacggtatatctagaaatgaacagcctgaaacctgaggacacggccgtctattcttgtaactccgaatactactggggccaggggacccaggtcaccgtctcctcaggtggaggcggttcaggcggaggtggctctggcggtggcggaagtggtggaggcggttcagatgtgcagctgcaggagtctgggggaggcttggtgcagcctggggggtctctgagactctcctgtgtaatctctggaatggacttcagtcacaaacccgcgggctggttccgccaggctccaggaaaagagcgcgagttcgtcgcttcgattacgactcgtgctagcacgcactatgcagactccgtgaagggccgattcaccatctccagagacaacgccaagaacacggtatatctagaaatgaacagcctgaaacctgaggacacggccgtctattcttgtaactccgaatactactggggccaggggacccaggtcaccgtctcctcataatga

SEQ ID NO: 21 - DNA, 705 bp, synthetic construct
  misc_feature 1..705: note = Polynucleotide sequence encoding ID25A construct
  source 1..705: mol_type = other DNA
gatgtgcagctgcaggagtctgggggaggcttggtgcagcctggggggtctctgagactctcctgtgtaatctctggaatggacttcagtcacaaacccgcgggctggttccgccaggctccaggaaaagagcgcgagttcgtcgcttcgattacgactcgtgctagcacgcactatgcagactccgtgaagggccgattcaccatctccagagacaacgccaagaacacggtatatctagaaatgaacagcctgaaacctgaggacacggccgtctattcttgtaactccgaatactactggggccaggggacccaggtcaccgtctcctcaggtggaggcggttcaaaaggcggaggtggctctgatgtgcagctgcaggagtctgggggaggcttggtgcagcctggggggtctctgagactctcctgtgtaatctctggaatggacttcagtcacaaacccgcgggctggttccgccaggctccaggaaaagagcgcgagttcgtcgcttcgattacgactcgtgctagcacgcactatgcagactccgtgaagggccgattcaccatctccagagacaacgccaagaacacggtatatctagaaatgaacagcctgaaacctgaggacacggccgtctattcttgtaactccgaatactactggggccaggggacccaggtcaccgtctcctcataatga

SEQ ID NO: 22 - DNA, 711 bp, synthetic construct
  misc_feature 1..711: note = Polynucleotide sequence encoding ID26A construct
  source 1..711: mol_type = other DNA
gatgtgcagctgcaggagtctgggggaggcttggtgcagcctggggggtctctgagactctcctgtgtaatctctggaatggacttcagtcacaaacccgcgggctggttccgccaggctccaggaaaagagcgcgagttcgtcgcttcgattacgactcgtgctagcacgcactatgcagactccgtgaagggccgattcaccatctccagagacaacgccaagaacacggtatatctagaaatgaacagcctgaaacctgaggacacggccgtctattcttgtaactccgaatactactggggccaggggacccaggtcaccgtctcctcaggtggaggcggttcagataaagacggcggaggtggctctgatgtgcagctgcaggagtctgggggaggcttggtgcagcctggggggtctctgagactctcctgtgtaatctctggaatggacttcagtcacaaacccgcgggctggttccgccaggctccaggaaaagagcgcgagttcgtcgcttcgattacgactcgtgctagcacgcactatgcagactccgtgaagggccgattcaccatctccagagacaacgccaagaacacggtatatctagaaatgaacagcctgaaacctgaggacacggccgtctattcttgtaactccgaatactactggggccaggggacccaggtcaccgtctcctcataatga

SEQ ID NO: 23 - DNA, 717 bp, synthetic construct
  misc_feature 1..717: note = Polynucleotide sequence encoding ID27A construct
  source 1..717: mol_type = other DNA
gatgtgcagctgcaggagtctgggggaggcttggtgcagcctggggggtctctgagactctcctgtgtaatctctggaatggacttcagtcacaaacccgcgggctggttccgccaggctccaggaaaagagcgcgagttcgtcgcttcgattacgactcgtgctagcacgcactatgcagactccgtgaagggccgattcaccatctccagagacaacgccaagaacacggtatatctagaaatgaacagcctgaaacctgaggacacggccgtctattcttgtaactccgaatactactggggccaggggacccaggtcaccgtctcctcaggtggaggcggttcagatgacgacgataaaggcggaggtggctctgatgtgcagctgcaggagtctgggggaggcttggtgcagcctggggggtctctgagactctcctgtgtaatctctggaatggacttcagtcacaaacccgcgggctggttccgccaggctccaggaaaagagcgcgagttcgtcgcttcgattacgactcgtgctagcacgcactatgcagactccgtgaagggccgattcaccatctccagagacaacgccaagaacacggtatatctagaaatgaacagcctgaaacctgaggacacggccgtctattcttgtaactccgaatactactggggccaggggacccaggtcaccgtctcctcataatga

SEQ ID NO: 24 - DNA, 708 bp, synthetic construct
  misc_feature 1..708: note = Polynucleotide sequence encoding ID28A construct
  source 1..708: mol_type = other DNA
gatgtgcagctgcaggagtctgggggaggcttggtgcagcctggggggtctctgagactctcctgtgtaatctctggaatggacttcagtcacaaacccgcgggctggttccgccaggctccaggaaaagagcgcgagttcgtcgcttcgattacgactcgtgctagcacgcactatgcagactccgtgaagggccgattcaccatctccagagacaacgccaagaacacggtatatctagaaatgaacagcctgaaacctgaggacacggccgtctattcttgtaactccgaatactactggggccaggggacccaggtcaccgtctcctcaaaaggtggaggcggttcaggcggaggtggctctaaggatgtgcagctgcaggagtctgggggaggcttggtgcagcctggggggtctctgagactctcctgtgtaatctctggaatggacttcagtcacaaacccgcgggctggttccgccaggctccaggaaaagagcgcgagttcgtcgcttcgattacgactcgtgctagcacgcactatgcagactccgtgaagggccgattcaccatctccagagacaacgccaagaacacggtatatctagaaatgaacagcctgaaacctgaggacacggccgtctattcttgtaactccgaatactactggggccaggggacccaggtcaccgtctcctcataatga

SEQ ID NO: 25 - DNA, 810 bp, synthetic construct
  misc_feature 1..810: note = Polynucleotide sequence encoding ID55F construct
  source 1..810: mol_type = other DNA
gaggtgcagctggtggagtctgggggaggattggttgagagtggggcctctctgagactctcctgtgtaacctctggacatatcttcaagttgtatggcatgggctggttccggcaggctcccgggaaggaccgtgagttcgtagcggctgttacatggaacggtccgagcacagagtacgcagactccgtgaagggccgattcaccatctccaaggacaacgacaggaacacgctgtatctgcaaatgaacagcctgaaacctgaggacacggccgtttattactgtgcacgaagttccataatgggaatatatactacccccgacagatacgaatactggggccaggggacccaagtcaccgtctcctcaggtggaggcggttcaggcggaggtggctctggcggtggcggaagtggtggcggtggatcagaggtgcagctggtggagtctgggggaggattggttgagagtggggcctctctgagactctcctgtgtaacctctggacatatcttcaagttgtatggcatgggctggttccggcaggctcccgggaaggaccgtgagttcgtagcggctgttacatggaacggtccgagcacagagtacgcagactccgtgaagggccgattcaccatctccaaggacaacgacaggaacacgctgtatctgcaaatgaacagcctgaaacctgaggacacggccgtttattactgtgcacgaagttccataatgggaatatatactacccccgacagatacgaatactggggccaggggacccaggtcaccgtctcctcataatga

SEQ ID NO: 26 - DNA, 783 bp, synthetic construct
  misc_feature 1..783: note = Polynucleotide sequence encoding ID57F construct
  source 1..783: mol_type = other DNA
gaggtgcagctggtggagtctgggggaggattggttgagagtggggcctctctgagactctcctgtgtaacctctggacatatcttcaagttgtatggcatgggctggttccggcaggctcccgggaaggaccgtgagttcgtagcggctgttacatggaacggtccgagcacagagtacgcagactccgtgaagggccgattcaccatctccaaggacaacgacaggaacacgctgtatctgcaaatgaacagcctgaaacctgaggacacggccgtttattactgtgcacgaagttccataatgggaatatatactacccccgacagatacgaatactggggccaggggacccaagtcaccgtctcctcaggtggaggcggttcaaaaggcggaggtggctctgaggtgcagctggtggagtctgggggaggattggttgagagtggggcctctctgagactctcctgtgtaacctctggacatatcttcaagttgtatggcatgggctggttccggcaggctcccgggaaggaccgtgagttcgtagcggctgttacatggaacggtccgagcacagagtacgcagactccgtgaagggccgattcaccatctccaaggacaacgacaggaacacgctgtatctgcaaatgaacagcctgaaacctgaggacacggccgtttattactgtgcacgaagttccataatgggaatatatactacccccgacagatacgaatactggggccaggggacccaggtcaccgtctcctcataatga

SEQ ID NO: 27 - DNA, 789 bp, synthetic construct
  misc_feature 1..789: note = Polynucleotide sequence encoding ID58F construct
  source 1..789: mol_type = other DNA
gaggtgcagctggtggagtctgggggaggattggttgagagtggggcctctctgagactctcctgtgtaacctctggacatatcttcaagttgtatggcatgggctggttccggcaggctcccgggaaggaccgtgagttcgtagcggctgttacatggaacggtccgagcacagagtacgcagactccgtgaagggccgattcaccatctccaaggacaacgacaggaacacgctgtatctgcaaatgaacagcctgaaacctgaggacacggccgtttattactgtgcacgaagttccataatgggaatatatactacccccgacagatacgaatactggggccaggggacccaagtcaccgtctcctcaggtggaggcggttcagataaagatggcggaggtggctctgaggtgcagctggtggagtctgggggaggattggttgagagtggggcctctctgagactctcctgtgtaacctctggacatatcttcaagttgtatggcatgggctggttccggcaggctcccgggaaggaccgtgagttcgtagcggctgttacatggaacggtccgagcacagagtacgcagactccgtgaagggccgattcaccatctccaaggacaacgacaggaacacgctgtatctgcaaatgaacagcctgaaacctgaggacacggccgtttattactgtgcacgaagttccataatgggaatatatactacccccgacagatacgaatactggggccaggggacccaggtcaccgtctcctcataatga

SEQ ID NO: 28 - DNA, 795 bp, synthetic construct
  misc_feature 1..795: note = Polynucleotide sequence encoding ID59F construct
  source 1..795: mol_type = other DNA
gaggtgcagctggtggagtctgggggaggattggttgagagtggggcctctctgagactctcctgtgtaacctctggacatatcttcaagttgtatggcatgggctggttccggcaggctcccgggaaggaccgtgagttcgtagcggctgttacatggaacggtccgagcacagagtacgcagactccgtgaagggccgattcaccatctccaaggacaacgacaggaacacgctgtatctgcaaatgaacagcctgaaacctgaggacacggccgtttattactgtgcacgaagttccataatgggaatatatactacccccgacagatacgaatactggggccaggggacccaagtcaccgtctcctcaggtggaggcggttcagacgatgacgataaaggcggaggtggctctgaggtgcagctggtggagtctgggggaggattggttgagagtggggcctctctgagactctcctgtgtaacctctggacatatcttcaagttgtatggcatgggctggttccggcaggctcccgggaaggaccgtgagttcgtagcggctgttacatggaacggtccgagcacagagtacgcagactccgtgaagggccgattcaccatctccaaggacaacgacaggaacacgctgtatctgcaaatgaacagcctgaaacctgaggacacggccgtttattactgtgcacgaagttccataatgggaatatatactacccccgacagatacgaatactggggccaggggacccaggtcaccgtctcctcataatga

SEQ ID NO: 29 - DNA, 786 bp, synthetic construct
  misc_feature 1..786: note = Polynucleotide sequence encoding ID60F construct
  source 1..786: mol_type = other DNA
gaggtgcagctggtggagtctgggggaggattggttgagagtggggcctctctgagactctcctgtgtaacctctggacatatcttcaagttgtatggcatgggctggttccggcaggctcccgggaaggaccgtgagttcgtagcggctgttacatggaacggtccgagcacagagtacgcagactccgtgaagggccgattcaccatctccaaggacaacgacaggaacacgctgtatctgcaaatgaacagcctgaaacctgaggacacggccgtttattactgtgcacgaagttccataatgggaatatatactacccccgacagatacgaatactggggccaggggacccaagtcaccgtctcctcaaaaggtggaggcggttcaggcggaggtggctctaaagaggtgcagctggtggagtctgggggaggattggttgagagtggggcctctctgagactctcctgtgtaacctctggacatatcttcaagttgtatggcatgggctggttccggcaggctcccgggaaggaccgtgagttcgtagcggctgttacatggaacggtccgagcacagagtacgcagactccgtgaagggccgattcaccatctccaaggacaacgacaggaacacgctgtatctgcaaatgaacagcctgaaacctgaggacacggccgtttattactgtgcacgaagttccataatgggaatatatactacccccgacagatacgaatactggggccaggggacccaggtcaccgtctcctcataatga

SEQ ID NO: 30 - DNA, 795 bp, synthetic construct; identical to SEQ ID NO: 28
  misc_feature 1..795: note = Polynucleotide sequence encoding ID56F construct
  source 1..795: mol_type = other DNA

SEQ ID NO: 31 - DNA, 60 bp, synthetic construct
  misc_feature 1..60: note = Polynucleotide sequence encoding linker used in constructs ID3A and ID55F
  source 1..60: mol_type = other DNA
ggtggtggtggttctggtggtggtggttctggtggtggtggttctggtggtggtggttct

SEQ ID NO: 32 - DNA, 33 bp, synthetic construct
  misc_feature 1..33: note = Polynucleotide sequence encoding linker used in constructs ID25A and ID57F
  source 1..33: mol_type = other DNA
ggtggtggtggttctaaaggtggtggtggttct

SEQ ID NO: 33 - DNA, 39 bp, synthetic construct
  misc_feature 1..39: note = Polynucleotide sequence encoding linker used in constructs ID26A and ID58F
  source 1..39: mol_type = other DNA
ggtggtggtggttctgataaagatggtggtggtggttct

SEQ ID NO: 34 - DNA, 45 bp, synthetic construct
  misc_feature 1..45: note = Polynucleotide sequence encoding linker used in constructs ID27A and ID59F
  source 1..45: mol_type = other DNA
ggtggtggtggttctgatgatgatgataaaggtggtggtggttct

SEQ ID NO: 35 - DNA, 36 bp, synthetic construct
  misc_feature 1..36: note = Polynucleotide sequence encoding linker used in constructs ID28A and ID60F
  source 1..36: mol_type = other DNA
aaaggtggtggtggttctggtggtggtggttctaaa

SEQ ID NO: 36 - DNA, 39 bp, synthetic construct
  misc_feature 1..39: note = Polynucleotide sequence encoding linker used in construct ID56F
  source 1..39: mol_type = other DNA
agaggtggtggtggttctagaggtggtggtggttctaga

SEQ ID NO: 37 - protein, 11 aa, synthetic construct
  REGION 1..11: note = Proposed chymotrypsin-labile linker
GGGGSYGGGGS

SEQ ID NO: 38 - protein, 235 aa, synthetic construct
  REGION 1..235: note = Polypeptide sequence of ID4A construct
DVQLQESGGGLVQPGGSLRLSCVISGMDFSHKPAGWFRQAPGKEREFVASITTRASTHYADSVKGRFTISRDNAKNTVYLEMNSLKPEDTAVYSCNSEYYWGQGTQVTVSSRGGGGSRGGGGSRDVQLQESGGGLVQPGGSLRLSCVISGMDFSHKPAGWFRQAPGKEREFVASITTRASTHYADSVKGRFTISRDNAKNTVYLEMNSLKPEDTAVYSCNSEYYWGQGTQVTVSS

SEQ ID NO: 39 - protein, 2210 aa, synthetic construct
  REGION 1..2210: note = Description of Artificial Sequence: Synthetic labile peptide linker
  REGION 1..10: note = MISC_FEATURE - This region may encompass 1-10 residues
  REGION 12..21: note = MISC_FEATURE - This region may encompass 1-10 residues
  REGION 23..32: note = MISC_FEATURE - This region may encompass 1-10 residues
  REGION 34..43: note = MISC_FEATURE - This region may encompass 1-10 residues
  REGION 45..54: note = MISC_FEATURE - This region may encompass 1-10 residues
  REGION 56..65: note = MISC_FEATURE - This region may encompass 1-10 residues
  REGION 67..76: note = MISC_FEATURE - This region may encompass 1-10 residues
  REGION 78..87: note = MISC_FEATURE - This region may encompass 1-10 residues
  REGION 89..98: note = MISC_FEATURE - This region may encompass 1-10 residues
  REGION 100..109: note = MISC_FEATURE - This region may encompass 1-10 residues
  VAR_SEQ 111
  REGION 112..121: note = MISC_FEATURE - This region may encompass 1-10 residues
  REGION 123..132: note = MISC_FEATURE - This region may encompass 1-10 residues
  REGION 134..143: note = MISC_FEATURE - This region may encompass 1-10 residues
  REGION 145..154: note = MISC_FEATURE - This region may encompass 1-10 residues
  REGION 156..165: note = MISC_FEATURE - This region may encompass 1-10 residues
  REGION 167..176: note = MISC_FEATURE - This region may encompass 1-10 residues
  REGION 178..187: note = MISC_FEATURE - This region may encompass 1-10 residues
  REGION 189..198: note = MISC_FEATURE - This region may encompass 1-10 residues
  REGION 200..209: note = MISC_FEATURE - This region may encompass 1-10 residues
  REGION 211..220: note = MISC_FEATURE - This region may encompass 1-10 residues
  REGION 222..231: note = MISC_FEATURE - This region may encompass 1-10 residues
  REGION 233..242: note = MISC_FEATURE - This region may encompass 1-10 residues
  REGION 244..253: note = MISC_FEATURE - This region may encompass 1-10 residues
  REGION 255..264: note = MISC_FEATURE - This region may encompass 1-10 residues
  REGION 266..275: note = MISC_FEATURE - This region may encompass 1-10 residues
  REGION 277..286: note = MISC_FEATURE - This region may encompass 1-10 residues
  REGION 288..297: note = MISC_FEATURE - This region may encompass 1-10 residues
  REGION 299..308: note = MISC_FEATURE - This region may encompass 1-10 residues
  REGION 310..319: note = MISC_FEATURE - This region may encompass 1-10 residues
  REGION 321..330: note = MISC_FEATURE - This region may encompass 1-10 residues
  VAR_SEQ 332
  REGION 333..342: note = MISC_FEATURE - This region may encompass 1-10 residues
  REGION 344..353: note = MISC_FEATURE - This region may encompass 1-10 residues
  REGION 355..364: note = MISC_FEATURE - This region may encompass 1-10 residues
  REGION 366..375: note = MISC_FEATURE - This region may encompass 1-10 residues
  REGION 377..386: note = MISC_FEATURE - This region may encompass 1-10 residues
  REGION 388..397: note = MISC_FEATURE - This region may encompass 1-10 residues
  REGION 399..408: note = MISC_FEATURE - This region may encompass 1-10 residues
  REGION 410..419: note = MISC_FEATURE - This region may encompass 1-10 residues
  REGION 421..430: note = MISC_FEATURE - This region may encompass 1-10 residues
  REGION 432..441: note = MISC_FEATURE - This region may encompass 1-10 residues
  REGION 443..452: note = MISC_FEATURE - This region may encompass 1-10 residues
  REGION 454..463: note = MISC_FEATURE - This region may encompass 1-10 residues
  REGION 465..474: note = MISC_FEATURE - This region may encompass 1-10 residues
  REGION 476..485: note = MISC_FEATURE - This region may encompass 1-10 residues
  REGION 487..496: note = MISC_FEATURE - This region may encompass 1-10 residues
  REGION 498..507: note = MISC_FEATURE - This region may encompass 1-10 residues
  REGION 509..518: note = MISC_FEATURE - This region may encompass 1-10 residues
  REGION 520..529: note = MISC_FEATURE - This region may encompass 1-10 residues
  REGION 531..540: note = MISC_FEATURE - This region may encompass 1-10 residues
  REGION 542..551: note = MISC_FEATURE - This region may encompass 1-10 residues
  VAR_SEQ 553
  REGION 554..563: note = MISC_FEATURE - This region may encompass 1-10 residues
  REGION 565..574: note = MISC_FEATURE - This region may encompass 1-10 residues
  REGION 576..585: note = MISC_FEATURE - This region may encompass 1-10 residues
  REGION 587..596: note = MISC_FEATURE - This region may encompass 1-10 residues
  REGION 598..607: note = MISC_FEATURE - This region may encompass 1-10 residues
  REGION 609..618: note = MISC_FEATURE - This region may encompass 1-10 residues
  REGION 620..629: note = MISC_FEATURE - This region may encompass 1-10 residues
  REGION 631..640: note = MISC_FEATURE - This region may encompass 1-10 residues
  REGION 642..651: note = MISC_FEATURE - This region may encompass 1-10 residues
  REGION 653..662: note = MISC_FEATURE - This region may encompass 1-10 residues
  REGION 664..673: note = MISC_FEATURE - This region may encompass 1-10 residues
  REGION 675..684: note = MISC_FEATURE - This region may encompass 1-10 residues
  REGION 686..695: note = MISC_FEATURE - This region may encompass 1-10 residues
  REGION 697..706: note = MISC_FEATURE - This region may encompass 1-10 residues
  REGION 708..717: note = MISC_FEATURE - This region may encompass 1-10 residues
  REGION 719..728: note = MISC_FEATURE - This region may encompass 1-10 residues
  REGION 730..739: note = MISC_FEATURE - This region may encompass 1-10 residues
  REGION 741..750: note = MISC_FEATURE - This region may encompass 1-10 residues
  REGION 752..761: note = MISC_FEATURE - This region may encompass 1-10 residues
  REGION 763..772: note = MISC_FEATURE - This region may encompass 1-10 residues
  VAR_SEQ 774
  REGION 775..784: note = MISC_FEATURE - This region may encompass 1-10 residues
  REGION 786..795: note = MISC_FEATURE - This region may encompass 1-10 residues
  REGION 797..806: note = MISC_FEATURE - This region may encompass 1-10 residues
  REGION 808..817: note = MISC_FEATURE - This region may encompass 1-10 residues
  REGION 819..828: note = MISC_FEATURE - This region may encompass 1-10 residues
  REGION 830..839: note = MISC_FEATURE - This region may encompass 1-10 residues
  REGION 841..850: note = MISC_FEATURE - This region may encompass 1-10 residues
  REGION 852..861: note = MISC_FEATURE - This region may encompass 1-10 residues
  REGION 863..872: note = MISC_FEATURE - This region may encompass 1-10 residues
  REGION 874..883: note = MISC_FEATURE - This region may encompass 1-10 residues
  REGION 885..894: note = MISC_FEATURE - This region may encompass 1-10 residues
  REGION 896..905: note = MISC_FEATURE - This region may encompass 1-10 residues
  REGION 907..916: note = MISC_FEATURE - This region may encompass 1-10 residues
  REGION 918..927: note = MISC_FEATURE - This region may encompass 1-10 residues
  REGION 929..938: note = MISC_FEATURE - This region may encompass 1-10 residues
  REGION 940..949: note = MISC_FEATURE - This region may encompass 1-10 residues
  REGION 951..960: note = MISC_FEATURE - This region may encompass 1-10 residues
  REGION 962..971: note = MISC_FEATURE - This region may encompass 1-10 residues
  REGION 973..982: note = MISC_FEATURE - This region may encompass 1-10 residues
  REGION 984..993: note = MISC_FEATURE - This region may encompass 1-10 residues
  VAR_SEQ 995
  REGION 996..1005: note = MISC_FEATURE - This region may encompass 1-10 residues
  REGION 1007..1016: note = MISC_FEATURE - This region may encompass 1-10 residues
  REGION 1018..1027: note = MISC_FEATURE - This region may encompass 1-10 residues
  REGION 1029..1038: note = MISC_FEATURE - This region may encompass 1-10 residues
  REGION 1040..1049: note = MISC_FEATURE - This region may encompass 1-10 residues
  REGION 1051..1060: note = MISC_FEATURE - This region may encompass 1-10 residues
  REGION 1062..1071: note = MISC_FEATURE - This region may encompass 1-10 residues
  REGION 1073..1082: note = MISC_FEATURE - This region may encompass 1-10 residues
  REGION 1084..1093: note = MISC_FEATURE - This region may encompass 1-10 residues
  REGION 1095..1104: note = MISC_FEATURE - This region may encompass 1-10 residues
  REGION 1106..1115: note = MISC_FEATURE - This region may encompass 1-10 residues
  REGION 1117..1126: note = MISC_FEATURE - This region may encompass 1-10 residues
  REGION 1128..1137: note = MISC_FEATURE - This region may encompass 1-10 residues
  REGION 1139..1148: note = MISC_FEATURE - This region may encompass 1-10 residues
  REGION 1150..1159: note = MISC_FEATURE - This region may encompass 1-10 residues
  REGION 1161..1170: note = MISC_FEATURE - This region may encompass 1-10 residues
  REGION 1172..1181: note = MISC_FEATURE - This region may encompass 1-10 residues
  REGION 1183..1192: note = MISC_FEATURE - This region may encompass 1-10 residues
  REGION 1194..1203: note = MISC_FEATURE - This region may encompass 1-10 residues
  REGION 1205..1214: note = MISC_FEATURE - This region may encompass 1-10 residues
  VAR_SEQ 1216
  REGION 1217..1226: note = MISC_FEATURE - This region may encompass 1-10 residues
  REGION 1228..1237: note = MISC_FEATURE - This region may encompass 1-10 residues
  REGION 1239..1248: note = MISC_FEATURE - This region may encompass 1-10 residues
  REGION 1250..1259: note = MISC_FEATURE - This region may encompass 1-10 residues
  REGION 1261..1270: note = MISC_FEATURE - This region may encompass 1-10 residues
  REGION 1272..1281: note = MISC_FEATURE - This region may encompass 1-10 residues
  REGION 1283..1292: note = MISC_FEATURE - This region may encompass 1-10 residues
  REGION 1294..1303: note = MISC_FEATURE - This region may encompass 1-10 residues
  REGION 1305..1314: note = MISC_FEATURE - This region may encompass 1-10 residues
  REGION 1316..1325: note = MISC_FEATURE - This region may encompass 1-10 residues
  REGION 1327..1336: note = MISC_FEATURE - This region may encompass 1-10 residues
  REGION 1338..1347: note = MISC_FEATURE - This region may encompass 1-10 residues
  REGION 1349..1358: note = MISC_FEATURE - This region may encompass 1-10 residues
  REGION 1360..1369: note = MISC_FEATURE - This region may encompass 1-10 residues
  REGION 1371..1380: note = MISC_FEATURE - This region may encompass 1-10 residues
  REGION 1382..1391: note = MISC_FEATURE - This region may encompass 1-10 residues
  REGION 1393..1402: note = MISC_FEATURE - This region may encompass 1-10 residues
  REGION 1404..1413: note = MISC_FEATURE - This region may encompass 1-10 residues
  REGION 1415..1424: note = MISC_FEATURE - This region may encompass 1-10 residues
  REGION 1426..1435: note = MISC_FEATURE - This region may encompass 1-10 residues
  VAR_SEQ 1437
  REGION 1438..1447: note = MISC_FEATURE - This region may encompass 1-10 residues
  REGION 1449..1458: note = MISC_FEATURE - This region may encompass 1-10 residues
  REGION 1460..1469: note = MISC_FEATURE - This region may encompass 1-10 residues
  REGION 1471..1480: note = MISC_FEATURE - This region may encompass 1-10 residues
  REGION 1482..1491: note = MISC_FEATURE - This region may encompass 1-10 residues
  REGION 1493..1502: note = MISC_FEATURE - This region may encompass 1-10 residues
  REGION 1504..1513: note = MISC_FEATURE - This region may encompass 1-10 residues
  REGION 1515..1524: note = MISC_FEATURE - This region may encompass 1-10 residues
  REGION 1526..1535: note = MISC_FEATURE - This region may encompass 1-10 residues
  REGION 1537..1546: note = MISC_FEATURE - This region may encompass 1-10 residues
  REGION 1548..1557: note = MISC_FEATURE - This region may encompass 1-10 residues
  REGION 1559..1568: note = MISC_FEATURE - This region may encompass 1-10 residues
  REGION 1570..1579: note = MISC_FEATURE - This region may encompass 1-10 residues
  REGION 1581..1590: note = MISC_FEATURE - This region may encompass 1-10 residues
  REGION 1592..1601: note = MISC_FEATURE - This region may encompass 1-10 residues
  REGION 1603..1612: note = MISC_FEATURE - This region may encompass 1-10 residues
  REGION 1614..1623: note = MISC_FEATURE - This region may encompass 1-10 residues
  REGION 1625..1634: note = MISC_FEATURE - This region may encompass 1-10 residues
  REGION 1636..1645: note = MISC_FEATURE - This region may encompass 1-10 residues
  REGION 1647..1656: note = MISC_FEATURE - This region may encompass 1-10 residues
  VAR_SEQ 1658
  REGION 1659..1668: note = MISC_FEATURE - This region may encompass 1-10 residues
  REGION 1670..1679: note = MISC_FEATURE - This region may encompass 1-10 residues
  REGION 1681..1690: note = MISC_FEATURE - This region may encompass 1-10 residues
  REGION 1692..1701: note = MISC_FEATURE - This region may encompass 1-10 residues
  REGION 1703..1712: note = MISC_FEATURE - This region may encompass 1-10 residues
  REGION 1714..1723: note = MISC_FEATURE - This region may encompass 1-10 residues
  REGION 1725..1734: note = MISC_FEATURE - This region may encompass 1-10 residues
  REGION 1736..1745: note = MISC_FEATURE - This region may encompass 1-10 residues
  REGION 1747..1756: note = MISC_FEATURE - This region may encompass 1-10 residues
  REGION 1758..1767: note = MISC_FEATURE - This region may encompass 1-10 residues
  REGION 1769..1778: note = MISC_FEATURE - This region may encompass 1-10 residues
  REGION 1780..1789: note = MISC_FEATURE - This region may encompass 1-10 residues
  REGION 1791..1800: note = MISC_FEATURE - This region may encompass 1-10 residues
  REGION 1802..1811: note = MISC_FEATURE - This region may encompass 1-10 residues
  REGION 1813..1822: note = MISC_FEATURE - This region may encompass 1-10 residues
  REGION 1824..1833: note = MISC_FEATURE - This region may encompass 1-10 residues
  REGION 1835..1844: note = MISC_FEATURE - This region may encompass 1-10 residues
  REGION 1846..1855: note = MISC_FEATURE - This region may encompass 1-10 residues
  REGION 1857..1866: note = MISC_FEATURE - This region may encompass 1-10 residues
  REGION 1868..1877: note = MISC_FEATURE - This region may encompass 1-10 residues
  VAR_SEQ 1879
  REGION 1880..1889: note = MISC_FEATURE - This region may encompass 1-10 residues
  REGION 1891..1900: note = MISC_FEATURE - This region may encompass 1-10 residues
  REGION 1902..1911: note = MISC_FEATURE - This region may encompass 1-10 residues
  REGION 1913..1922: note = MISC_FEATURE - This region may encompass 1-10 residues
  REGION 1924..1933: note = MISC_FEATURE - This region may encompass 1-10 residues
  REGION 1935..1944: note = MISC_FEATURE - This region may encompass 1-10 residues
  REGION 1946..1955: note = MISC_FEATURE - This region may encompass 1-10 residues
  REGION 1957..1966: note = MISC_FEATURE - This region may encompass 1-10 residues
  REGION 1968..1977: note = MISC_FEATURE - This region may encompass 1-10 residues
  REGION 1979..1988: note = MISC_FEATURE - This region may encompass 1-10 residues
  REGION 1990..1999: note = MISC_FEATURE - This region may encompass 1-10 residues
  REGION 2001..2010: note = MISC_FEATURE - This region may encompass 1-10 residues
  REGION 2012..2021: note = MISC_FEATURE - This region may encompass 1-10 residues
  REGION 2023..2032: note = MISC_FEATURE - This region may encompass 1-10 residues
  REGION 2034..2043: note = MISC_FEATURE - This region may encompass 1-10 residues
  REGION 2045..2054: note = MISC_FEATURE - This region may encompass 1-10 residues
  REGION 2056..2065: note = MISC_FEATURE - This region may encompass 1-10 residues
  REGION 2067..2076: note = MISC_FEATURE - This region may encompass 1-10 residues
  REGION 2078..2087: note = MISC_FEATURE - This region may encompass 1-10 residues
  REGION 2089..2098: note = MISC_FEATURE - This region may encompass 1-10 residues
  VAR_SEQ 2100
  REGION 2101..2110: note = MISC_FEATURE - This region may encompass 1-10 residues
  REGION 2112..2121: note = MISC_FEATURE - This region may encompass 1-10 residues
  REGION 2123..2132: note = MISC_FEATURE - This region may encompass 1-10 residues
  REGION 2134..2143: note = MISC_FEATURE - This region may encompass 1-10 residues
  REGION 2145..2154: note = MISC_FEATURE - This region may encompass 1-10 residues
  REGION 2156..2165: note = MISC_FEATURE - This region may encompass 1-10 residues
  REGION 2167..2176: note = MISC_FEATURE - This region may encompass 1-10 residues
  REGION 2178..2187: note = MISC_FEATURE - This region may encompass 1-10 residues
  REGION 2189..2198: note = MISC_FEATURE - This region may encompass 1-10 residues
  REGION 2200..2209: note = MISC_FEATURE - This region may encompass 1-10 residues
  REGION 1..2210: note = MISC_FEATURE - This sequence may encompass 1-10 "(GaS)x-B-(GbS)y" repeating units wherein a is 1 to 10, b is 1 to 10 x is 1 to 10, and y is 1 to 10.
  REGION 1..2210: note = See specification as filed for detailed description of substitutions and preferred embodiments
GGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSXGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSXGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSXGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSXGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSXGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSXGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSXGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSXGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSXGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSXGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGS

SEQ ID NO: 40 - protein, 112 aa, synthetic construct
  REGION 1..112: note = Description of Artificial Sequence: Synthetic labile peptide linker
  VAR_SEQ 1
  REGION 2..11: note = MISC_FEATURE - This region may encompass 1-10 residues
  REGION 13..22: note = MISC_FEATURE - This region may encompass 1-10 residues
  REGION 24..33: note = MISC_FEATURE - This region may encompass 1-10 residues
  REGION 35..44: note = MISC_FEATURE - This region may encompass 1-10 residues
  REGION 46..55: note = MISC_FEATURE - This region may encompass 1-10 residues
  REGION 57..66: note = MISC_FEATURE - This region may encompass 1-10 residues
  REGION 68..77: note = MISC_FEATURE - This region may encompass 1-10 residues
  REGION 79..88: note = MISC_FEATURE - This region may encompass 1-10 residues
  REGION 90..99: note = MISC_FEATURE - This region may encompass 1-10 residues
  REGION 101..110: note = MISC_FEATURE - This region may encompass 1-10 residues
  VAR_SEQ 112
  REGION 1..112: note = See specification as filed for detailed description of substitutions and preferred embodiments
XGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSX

SEQ ID NO: 41 - protein, 23 aa, synthetic construct
  REGION 1..23: note = Description of Artificial Sequence: Synthetic labile peptide linker
  REGION 1..10: note = MISC_FEATURE - This region may encompass 1-10 residues
  VAR_SEQ 12
  REGION 13..22: note = MISC_FEATURE - This region may encompass 1-10 residues
  REGION 1..23: note = See specification as filed for detailed description of substitutions and preferred embodiments
GGGGGGGGGGSXGGGGGGGGGGS

SEQ ID NO: 42 - protein, 24 aa, synthetic construct
  REGION 1..24: note = Description of Artificial Sequence: Synthetic labile peptide linker
  VAR_SEQ 1
  REGION 2..11: note = MISC_FEATURE - This region may encompass 1-10 residues
  REGION 13..22: note = MISC_FEATURE - This region may encompass 1-10 residues
  VAR_SEQ 24
  REGION 1..24: note = See specification as filed for detailed description of substitutions and preferred embodiments
XGGGGGGGGGGSGGGGGGGGGGSX

(19) United States
(12) Patent Application Publication
(10) Pub. No.: US 2025/0388655 A1
(43) Pub. Date: Dec. 25, 2025

(54) PEPTIDE CONSTRUCT HAVING A PROTEASE-CLEAVABLE LINKER

(71) Applicant: Sorriso Pharmaceuticals, Inc., Sandy, UT (US)

(72) Inventors: Scott CROWE, Babraham (GB); Mike WEST, Babraham (GB); Kevin ROBERTS, Babraham (GB)

(21) Appl. No.: 19/025,399

(22) Filed: Jan. 16, 2025

Related U.S. Application Data
(63) Continuation of application No. 17/196,498, filed on Mar. 9, 2021, now Pat. No. 12,234,279.
Application No. 17/196,498 is a continuation of application No. 15/717,174, filed on Sep. 27, 2017, now abandoned.
Application No. 15/717,174 is a continuation of application No. PCT/EP2016/057022, filed on Mar. 31, 2016.

(30) Foreign Application Priority Data
Mar. 31, 2015 (EP) 15162112.5
Mar. 31, 2015 (EP) 15162114.1
Mar. 31, 2015 (EP) 15162115.8
Jan. 21, 2016 (EP) 16152320.4

(51) Int. Cl. C07K 16/12; C07K 16/24

(52) U.S. Cl. CPC C07K 16/1282; C07K 16/241; C07K 2317/56; C07K 2317/64; C07K 2317/94; C07K 2319/50

(57) ABSTRACT

There is provided inter alia a construct suitable for oral administration comprising a first polypeptide and a second polypeptide connected by a labile peptide linker, wherein the labile peptide linker is labile to one or more proteases present in the intestinal tract and wherein the first and second polypeptides are substantially resistant to said one or more proteases.

### CROSS-REFERENCE TO RELATED APPLICATIONS

The present application is a continuation application of U.S. application Ser. No. 17/196,498, filed Mar. 9, 2021, which is a continuation application of U.S. application Ser. No. 15/717,174, filed Sep. 27, 2017, now abandoned, which is a continuation application of International Patent Application Serial No. PCT/EP2016/057022, filed Mar. 31, 2016, which claims priority from EP 15162114.1 filed Mar. 31, 2015, EP 15162115.8 filed Mar. 31, 2015, EP 15162112.5 filed Mar. 31, 2015 and EP 16152320.4 filed Jan. 21, 2016, the contents of each of which are incorporated herein by reference in their entirety.

### FIELD OF THE INVENTION

The present invention relates to constructs suitable for oral administration comprising polypeptides connected by a labile peptide linker as well as to pharmaceutical compositions comprising such constructs. The present invention also relates to methods for preparing such constructs, methods which assay the lability of such constructs, methods which utilise such constructs, nucleic acids encoding such constructs, cDNA and vectors comprising nucleic acids encoding such constructs, host cells expressing or capable of expressing such constructs and to uses of such constructs or pharmaceutical compositions.

### SEQUENCE LISTING

The instant application contains a Sequence Listing which has been submitted electronically in XML format and is hereby incorporated by reference in its entirety. Said XML copy, created on Sep. 5, 2025, is named “60790-704.303.xml” and is 163,840 bytes in size.

### BACKGROUND OF THE INVENTION

Constructs comprising two or more polypeptides are a class of biomolecules with multi-functional properties. By genetically fusing two or more polypeptides together, the resultant construct may obtain many distinct functions derived from each component polypeptide. Such constructs have been utilised in biological research for many purposes such as protein purification and imaging. Such constructs have also become an important category of biopharmaceuticals. An effective construct requires a suitable linker. Direct fusion of polypeptides without a linker may lead to many undesirable outcomes including misfolding of the fused polypeptides, low yield in polypeptide production or impaired bioactivity. Therefore, the selection or rational design of a linker to join polypeptides is an important area in recombinant polypeptide technology.

Many constructs incorporate linkers which are relatively stable for the purposes of in vivo delivery or recombinant production. Stable linkers covalently join functional domains together to act as one molecule throughout the in vivo or recombinant production processes. A stable linkage between polypeptides may provide many advantages such as a prolonged plasma half-life and resistance to cleavage by host organism proteases. However, stable linkers also have several potential drawbacks including steric hindrance between polypeptides, decreased bioactivity, and altered biodistribution (Chen et al. 2013 Adv Drug Deliv Rev. 65(10):1357-1369). It would be advantageous therefore to release free polypeptides from a construct in vivo and thereby potentially reduce steric hindrance, improve bioactivity or achieve independent functions of individual polypeptides.

In the context of polypeptides having effects in the intestinal tract, such release would ideally take place in the intestinal tract after oral administration. It may be preferable that such release takes place only in one or a number of specific locations within the intestinal tract.

Constructs of the present invention may, in at least some embodiments, have one or more of the following advantages compared to substances of the prior art:
(i) increased suitability for oral administration;
(ii) increased suitability for local delivery to the intestinal tract following oral administration;
(iii) ability to target (i.e. cleave the construct into component polypeptides) at one or more specific regions of the intestinal tract;
(iv) increased convenience in incorporating two or more separable polypeptides in one recombinant product;
(v) improved treatment and/or prevention of gastrointestinal tract infection or autoimmune and/or inflammatory diseases;
(vi) increased suitability for expression, in a heterologous host such as bacteria (e.g. Escherichia coli) and/or mammalian cells and/or a yeast or mould (e.g. those belonging to the genera Aspergillus, Saccharomyces, Kluyveromyces, Hansenula or Pichia, such as Saccharomyces cerevisiae or Pichia pastoris);
(vii) increased stability to protease degradation during production (for example resistance to yeast, mould or mammalian cell proteases);
(viii) improved folding of polypeptides;
(ix) improved yield during recombinant production;
(x) improved bioactivity and/or biodistribution;
(xi) suitability for, and improved properties for, use in a pharmaceutical;
(xii) suitability for, and improved properties for, use in a functional food.

### SUMMARY OF THE INVENTION

The present inventors have produced surprisingly advantageous constructs suitable for oral administration comprising a first polypeptide and a second polypeptide connected by a labile peptide linker. These constructs are particularly advantageous due to their convenience of production and their ability to release free component polypeptides within the intestinal tract.

In one embodiment, the free component polypeptides may be released in one or more specific regions of the intestinal tract.

It may be expected that these constructs have particular utility in the prevention or treatment of diseases of the GIT such as autoimmune and/or inflammatory disease such as inflammatory bowel disease, or in the prevention or treatment of infection from an intestinal tract resident pathogenic microbe.

The present invention provides a construct suitable for oral administration comprising a first polypeptide and a second polypeptide connected by a labile peptide linker, wherein the labile peptide linker is labile to one or more proteases present in the intestinal tract and wherein the first and second polypeptides are substantially resistant to said one or more proteases. Also provided are related compositions, methods and nucleic acids relating to the inventive construct.

### DESCRIPTION OF THE FIGURES

FIG. 1—Stained PAGE gel demonstrating non-lability of ID3A using the Trypsin Protease Assay

FIG. 2—Stained PAGE gel demonstrating lability of ID25A and ID26A using the Trypsin Protease Assay

FIG. 3—Stained PAGE gel demonstrating lability of ID27A and ID28A using the Trypsin Protease Assay

FIG. 4—Stained PAGE gel demonstrating lability of ID3A, ID25A, ID26A, ID27A and ID28A using the Faecal Protease Assay

FIG. 5—Stained PAGE gel demonstrating lability of ID55F, ID56F, ID57F, ID58F, ID59F and ID60F using the Faecal Protease Assay

FIG. 6—Stained PAGE gels demonstrating storage stability of ID3A and ID25A

FIG. 7—Stained PAGE gel demonstrating storage stability of ID26A, ID27A and ID28A

FIG. 8—Stained PAGE gel demonstrating storage stability of ID4A

### DESCRIPTION OF THE SEQUENCES

SEQ ID NO: 1—Polypeptide sequence of linker used in constructs ID3A and ID55F
SEQ ID NO: 2—Polypeptide sequence of linker used in constructs ID25A and ID57F
SEQ ID NO: 3—Polypeptide sequence of linker used in constructs ID26A and ID58F
SEQ ID NO: 4—Polypeptide sequence of linker used in constructs ID27A and ID59F
SEQ ID NO: 5—Polypeptide sequence of linker used in constructs ID28A and ID60F
SEQ ID NO: 6—Polypeptide sequence of linker used in construct ID56F
SEQ ID NO: 7—Polypeptide sequence of ID3A construct
SEQ ID NO: 8—Polypeptide sequence of ID25A construct
SEQ ID NO: 9—Polypeptide sequence of ID26A construct
SEQ ID NO: 10—Polypeptide sequence of ID27A construct
SEQ ID NO: 11—Polypeptide sequence of ID28A construct
SEQ ID NO: 12—Polypeptide sequence of ID55F construct
SEQ ID NO: 13—Polypeptide sequence of ID57F construct
SEQ ID NO: 14—Polypeptide sequence of ID58F construct
SEQ ID NO: 15—Polypeptide sequence of ID59F construct
SEQ ID NO: 16—Polypeptide sequence of ID60F construct
SEQ ID NO: 17—Polypeptide sequence of ID56F construct
SEQ ID NO: 18—Polypeptide sequence of ID1A ICVD
SEQ ID NO: 19—Polypeptide sequence of ID5F ICVD
SEQ ID NO: 20—Polynucleotide sequence encoding ID3A construct
SEQ ID NO: 21—Polynucleotide sequence encoding ID25A construct
SEQ ID NO: 22—Polynucleotide sequence encoding ID26A construct
SEQ ID NO: 23—Polynucleotide sequence encoding ID27A construct
SEQ ID NO: 24—Polynucleotide sequence encoding ID28A construct
SEQ ID NO: 25—Polynucleotide sequence encoding ID55F construct
SEQ ID NO: 26—Polynucleotide sequence encoding ID57F construct
SEQ ID NO: 27—Polynucleotide sequence encoding ID58F construct
SEQ ID NO: 28—Polynucleotide sequence encoding ID59F construct
SEQ ID NO: 29—Polynucleotide sequence encoding ID60F construct
SEQ ID NO: 30—Polynucleotide sequence encoding ID56F construct
SEQ ID NO: 31—An exemplary polynucleotide sequence which would encode linker used in constructs ID3A and ID55F
SEQ ID NO: 32—An exemplary polynucleotide sequence which would encode linker used in constructs ID25A and ID57F
SEQ ID NO: 33—An exemplary polynucleotide sequence which would encode linker used in constructs ID26A and ID58F
SEQ ID NO: 34—An exemplary polynucleotide sequence which would encode linker used in constructs ID27A and ID59F
SEQ ID NO: 35—An exemplary polynucleotide sequence which would encode linker used in constructs ID28A and ID60F
SEQ ID NO: 36—An exemplary polynucleotide sequence which would encode linker used in construct ID56F
SEQ ID NO: 37—Proposed chymotrypsin-labile linker
SEQ ID NO: 38—Polypeptide sequence of ID4A construct

### DETAILED DESCRIPTION OF THE INVENTION

### Labile Peptide Linkers

The construct of the invention comprises a labile peptide linker, which connects the first and second polypeptides. In one embodiment of the invention, the labile peptide linker can be engineered such that it resists cleavage by proteases to a desired extent and/or is only cleaved upon exposure to a specific area of the intestinal tract. For example, if a construct is recombinantly produced in a host such as yeast, trypsin-like proteases produced by the yeast may cleave the recombinant construct product. This may result in difficulties in purification and cause regulatory, clinical and commercial complications. Similarly, if for example the first polypeptide is a toxin, the second polypeptide of the construct may act to ‘quench’ the effects of the toxin until it is released at a suitable, target location.

This can be achieved according to one embodiment of the invention by incorporating shielding residues into the labile peptide linker flanking the labile site(s). Shielding residues flank the labile site(s) of the labile peptide linker and reduce the lability thereof. Cleavage resistance can also be increased by positioning the labile site(s) closer to or at the periphery of the labile peptide linker. This concept is referred to as a “shielded labile site” and provides controlled lability.

In a further embodiment of the invention, the labile peptide linker can be engineered such that it is highly labile to cleavage by intestinal tract proteases, thereby quickly releasing the constituent first and second polypeptides of the construct after oral administration. This is achieved by incorporating one or more labile sites into the labile peptide linker such that the labile site is exposed for proteolysis, for example by positioning the labile site(s) substantially centrally in the labile peptide linker and/or by the labile site not being shielded substantially by flanking residues. This concept is referred to as a “non-shielded labile site”.

Suitably the labile peptide linker has a length of at least 3, such as at least 4, such as at least 5, such as at least 6, such as at least 7, such as at least 8, such as at least 9, such as at least 10 residues. Suitably the labile peptide linker has a length of no greater than 40, such as no greater than 35, such as no greater than 30, such as no greater than 25, such as no greater than 20, such as no greater than 15 residues.

Incorporation of a P residue into the labile peptide linker of the construct of the invention is expected to substantially prevent cleavage of the labile peptide linker. Suitably the labile peptide linker does not comprise any P residues.

### Trypsin Labile Sites

### Shielded Trypsin Labile Sites

Suitably the labile peptide linker of the construct of the invention comprises a cleavage site for trypsin or a trypsin-like protease. Suitably the labile peptide linker comprises at least 1, such as at least 2, such as at least 3, such as at least 4, such as at least 5, such as at least 6, such as at least 7, such as at least 8, such as at least 9, such as at least 10 K residues. Suitably the labile peptide linker comprises at least 1, such as at least 2, such as at least 3, such as at least 4, such as at least 5, such as at least 6, such as at least 7, such as at least 8, such as at least 9, such as at least 10 R residues. Preferably the cleavage site(s) is/are one or more K residue(s).

Shielding residues in the case of a trypsin or trypsin-like protease labile site may be D or E. Suitably the labile peptide linker comprises one or more shielding residues selected from the list consisting of D or E.

Suitably all K or R residues comprised within the labile peptide linker have at least 1, such as at least 2, such as at least 3, such as at least 4, such as at least 5 shielding residues on their N-terminal side, wherein the shielding residues are selected from the list consisting of: D and E. Suitably all K or R residues comprised within the labile peptide linker have 1 to 5, 1 to 4, 1 to 3, 1 to 2, 2 to 5, 2 to 4, 2 to 3, 3 to 5, 3 to 4, 4 to 5 shielding residues on their N-terminal side, wherein the shielding residues are selected from the list consisting of: D and E.

Suitably all K or R residues comprised within the labile peptide linker have and at least 1, such as at least 2, such as at least 3, such as at least 4, such as at least 5 shielding residues on their C-terminal side, wherein the shielding residues are selected from the list consisting of: D and E. Suitably all K or R residues comprised within the labile peptide linker have 1 to 5, 1 to 4, 1 to 3, 1 to 2, 2 to 5, 2 to 4, 2 to 3, 3 to 5, 3 to 4, 4 to 5 shielding residues on their C-terminal side, wherein the shielding residues are selected from the list consisting of: D and E.

Suitably all K and R residues have at least one shielding residue adjacent to them, suitably followed by one or more further contiguous shielding residues. Suitably the shielding residues occur on one or both sides of one or more of the K or R residues.

Suitably the labile peptide linker comprises or more suitably consists of a polypeptide sequence of the format:

[-(GₐS)ₓ-U_{c}-B-U′_{d}-(G_{b}S)_{y}-]_{z}

wherein
a is 1 to 10;
b is 1 to 10;
U is D or E;
U′ is or E;
c is 0 to 7;
d is 0 to 7;
x is 1 to 10;
y is 1 to 10
z is 1 to 10 and
B is K or R.

Suitably a is 2 to 5, more suitably a is 4. Suitably b is 2 to 5, more suitably b is 4. Suitably x is 1 to 5, more suitably x is 1. Suitably y is 1 to 5, more suitably y is 1. Suitably z is 1 to 3, more suitably z is 1. Suitably, B is K. Suitably, U if present, is D. Suitably, U′ if present, is D. In one embodiment c is 1 and d is 1. In another embodiment c is 0 and d is 0. In a further embodiment c is 4 and d is 0. Suitably both U and U′ are each individually D and c and d are both 1.

Suitably the labile peptide linker comprises or more suitably consists of a polypeptide sequence of the format:

-B-(GₐS)ₓ-B′-

wherein
a is 1 to 10;
x is 1 to 10;
B is K or R and
B′ is K or R.

In one embodiment, a is 2 to 5, more suitably a is 4. In a further embodiment x is 1 to 5. More suitably, x is 2. Suitably, B is K. Suitably, B′ is K.

Suitably the labile peptide linker comprises or more suitably consists of a polypeptide sequence of the format:

-B-(GₐS)ₓ-B′-(G_{b}S)_{y}-B″-

wherein
a is 1 to 10;
b is 1 to 10;
x is 1 to 10;
y is 1 to 10;
B is K or R
B′ is K or R and
B″ is K or R.

In one embodiment, a is 2 to 5, more suitably a is 4. In one embodiment, b is 2 to 5, more suitably b is 4. In a further embodiment x is 1 to 5. More suitably, x is 2. In a further embodiment y is 1 to 5. More suitably, y is 2. Suitably, B is K. Suitably, B′ is K. Suitably, B″ is K.

### Non-Shielded Trypsin Labile Sites

Suitably the labile peptide linker of the construct of the invention comprises a cleavage site for trypsin or a trypsin-like protease. Suitably the labile peptide linker comprises at least 1, such as at least 2, such as at least 3, such as at least 4, such as at least 5, such as at least 6, such as at least 7, such as at least 8, such as at least 9, such as at least 10 K residues. Suitably the labile peptide linker comprises at least 1, such as at least 2, such as at least 3, such as at least 4, such as at least 5, such as at least 6, such as at least 7, such as at least 8, such as at least 9, such as at least 10 R residues. Preferably the cleavage site(s) is/are one or more K residue(s).

Suitably all K or R residues comprised within the labile peptide linker have at least 1, such as at least 2, such as at least 3, such as at least 4, such as at least 5 non-shielding residues on their N-terminal side wherein the non-shielding residues are selected from the list consisting of: C, A, S, N, G, L, I, V, T, M, F, Y, H, W and Q; more suitably A, G, L, I, V, M, S and T; more suitably A, G, L, I, V and S; more suitably G and S. Suitably all K or R residues comprised within the labile peptide linker have 1 to 5, 1 to 4, 1 to 3, 1 to 2, 2 to 5, 2 to 4, 2 to 3, 3 to 5, 3 to 4, 4 to 5 non-shielding residues on their N-terminal side, wherein the shielding residues are selected from the list consisting of: C, A, S, N, G, L, I, V, T, M, F, Y, H, W and Q; more suitably A, G, L, I, V, M, S and T; more suitably A, G, L, I, V and S; more suitably G and S.

Suitably all K or R residues comprised within the labile peptide linker have at least 1, such as at least 2, such as at least 3, such as at least 4, such as at least 5 non-shielding residues on their C-terminal side, wherein the non-shielding residues are selected from the list consisting of: C, A, S, N, G, L, I, V, T, M, F, Y, H, W and Q; more suitably A, G, L, I, V, M, S and T; more suitably A, G, L, I, V and S; more suitably G and S. Suitably all K or R residues comprised within the labile peptide linker have 1 to 5, 1 to 4, 1 to 3, 1 to 2, 2 to 5, 2 to 4, 2 to 3, 3 to 5, 3 to 4, 4 to 5 non-shielding residues on their C-terminal side, wherein the shielding residues are selected from the list consisting of: C, A, S, N, G, L, I, V, T, M, F, Y, H, W and Q; more suitably A, G, L, I, V, M, S and T; more suitably A, G, L, I, V and S; more suitably G and S.

Suitably all K and R residues have at least one non-shielding residue adjacent to them, suitably followed by one or more further contiguous non-shielding residues. Suitably the non-shielding residues occur on one or both sides of one or more of the K or R residues.

Suitably the labile peptide linker does not comprise any D or E residues. Suitably the labile peptide linker consists of residues selected from the list consisting of C, A, S, N, G, L, I, V, T, M, F, Y, H, K, R, W and Q; more suitably A, G, L, I, V, M, S, T, K and R residues; more suitably S, G, K and R residues.

Suitably the labile peptide linker comprises or more suitably consists of a polypeptide sequence of the format:

[-(GₐS)ₓ-B-(G_{b}S)_{y}-]_{z}

wherein
a is 1 to 10;
b is 1 to 10;
x is 1 to 10;
y is 1 to 10
z is 1 to 10 and
B is K or R.

Suitably a is 2 to 5, more suitably a is 4. Suitably b is 2 to 5, more suitably b is 4. Suitably x is 1 to 5, more suitably x is 1. Suitably y is 1 to 5, more suitably y is 1. Suitably z is 1 to 3, more suitably z is 1. Suitably B is K.

Suitably the labile peptide linker comprises or more suitably consists of a polypeptide sequence of the format:

[-(G₄S)ₓ-B-(G₄S)_{y}-]_{z}

wherein
x is 1 to 10;
y is 1 to 10
z is 1 to 10 and
B is K or R.

Suitably x is 1 to 5, more suitably x is 1. Suitably y is 1 to 5, more suitably y is 1. Suitably z is 1 to 3, more suitably, z is 1. Suitably B is K.

### Chymotrypsin Labile Sites

### Non-Shielded Chymotrypsin Labile Sites

Alternatively, or in addition to trypsin labile sites, the labile peptide linker of the construct of the invention comprises a cleavage site for chymotrypsin or a chymotrypsin-like protease. Suitably the labile peptide linker comprises at least 1, such as at least 2, such as at least 3, such as at least 4, such as at least 5, such as at least 6, such as at least 7, such as at least 8, such as at least 9, such as at least 10 residues selected from the list consisting of W, F, Y, L and M; more suitably W, F and Y. Suitably the labile peptide linker consists of residues selected from the list consisting of S, G, W, F, Y, L and M; such as S, G, W, F and Y.

Suitably the labile peptide linker comprises or more suitably consists of a polypeptide sequence of the format:

[-(GₐS)ₓ-J-(G_{b}S)_{y}-]_{z}

wherein
   a is 1 to 10;
   b is 1 to 10;
   x is 1 to 10;
   y is 1 to 10
   z is 1 to 10 and
   J is W, F, Y, L or M; such as W, F or Y.

In one embodiment a is 2 to 5, in a further embodiment, b is 2 to 5, in a further embodiment x is 1 to 5, in a further embodiment, y is 1 to 5, in a further embodiment z is 1 to 3. Suitably x is 1, y is 1 and z is 1.

Suitably the labile peptide linker comprises or more suitably consists of a polypeptide sequence of the format:

[-(G₄S)ₓ-J-(G₄S)_{y}-]_{z}

wherein
   x is 1 to 10;
   y is 1 to 10
   z is 1 to 10 and
   J is W, F, Y, L or M; such as W, F or Y.

In one embodiment, x is 1 to 5, in a further embodiment, y is 1 to 5, in a further embodiment z is 1 to 3. Suitably x is 1, y is 1 and z is 1.

### MMP Labile Sites

In one embodiment, the labile peptide linker of the construct of the invention comprises a cleavage site for MMP3, MMP10 or MMP12.

### Lability

The lability of a construct, and therefore the utility of a construct as a controlled-lability or highly labile construct can be assayed using the Faecal Protease Assay, the Trypsin Protease Assay and/or the Chymotrypsin Protease Assay defined in Examples 1 to 3.

Suitably at least 20%, such as at least 40%, such as at least 60%, such as at least 80%, such as at least 90%, such as at least 100% by mass of the construct remains uncleaved after at least 10, such as at least 20, such as at least 30, such as at least 40, such as at least 50, such as at least 60, such as at least 70, such as at least 80, such as at least 90, such as at least 100, such as at least 110, such as at least 120, such as at least 130, such as at least 140, such as at least 150, such as at least 160 minutes after mixing in the Faecal Protease Assay and/or Trypsin Protease Assay and/or Chymotrypsin Protease Assay.

Alternatively, at least 60%, such as at least 80%, such as at least 90%, such as at least 100% of the construct is cleaved after no more than 5, such as no more than 4, such as no more than 3, such as no more than 2, such as no more than 1 minute, after mixing in the Faecal Protease Assay and/or Trypsin Protease Assay and/or Chymotrypsin Protease Assay.

In one aspect of the invention there is provided a method of assaying the lability of a construct of the invention, comprising the steps of: (a) incubating the construct in a solution comprising trypsin, a solution comprising chymotrypsin, faecal supernatant, small intestinal fluid or a solution comprising enteropeptidase (such as by performing the Trypsin Protease Assay, the Chymotrypsin Protease Assay or the Faecal Protease Assay) then (b) ascertaining the proportion of cleaved construct after one or more periods of incubation.

In a further aspect of the invention there is provided a method of delivering a monomeric antibody or a monomeric antigen binding fragment thereof to a targeted region of the intestinal tract, comprising the steps of: (a) performing the method of assaying construct lability described above, (b) selecting a construct with an appropriate level of lability for the targeted region of the intestinal tract, (c) producing the selected construct with an enterically coated packaging then (d) administering the packaged selected construct to a subject.

In a further aspect of the invention there is provided a method of preparing a product comprising a construct of the invention which has been selected, the method comprising adding the selected construct into the product, wherein the selected construct is selected and produced by a method comprising the steps of: (a) performing the method of assaying construct lability described above then (b) selecting a construct with an appropriate level of lability for the targeted region of the intestinal tract.

### Stability

Various organisms may be used to express recombinant polypeptides. Commonly used expression organisms include yeast, mould and mammalian cells. However, many of these expression organisms also produce proteases, such as trypsin-like proteases, which may cleave the expressed recombinant polypeptide. If the expressed polypeptide incorporates a peptide linker which is labile to one or more proteases present in the intestinal tract, then this peptide linker may undesirably also be labile to proteases produced by the expression organism, thus preventing effective expression of intact polypeptide.

It is advantageous for the labile peptide linker to be substantially non-labile to enzymes produced by the recombinant host used to produce the construct. Suitably the labile peptide linker is substantially resistant to proteases produced by a recombinant host such as bacteria such as E. coli or such as a yeast or mould belonging to the genera Aspergillus, Saccharomyces, Kluyveromyces, Hansenula or Pichia; such as Saccharomyces cerevisiae or Pichia pastoris. Suitably the recombinant host is a yeast. Suitably the recombinant host is a mould. Suitably the yeast belongs to the genera Saccharomyces, Kluyveromyces, Hansenula or Pichia. Further examples of yeasts are those belonging to the genera Candida and Torulopsis. Suitably the mould belongs to the genus Aspergillus. Further examples of moulds are those belonging to the genera Acremonium, Alternaria, Chrysosporium, Cladosporium, Dictyostelium, Fusarium, Mucor, Penicillium, Rhizopus, Stachybotrys, Trichoderma and Trichophyton. Suitably the labile peptide linker is substantially resistant to proteases produced by a recombinant host for at least 2, such as at least 4, such as at least 9, such as at least 14, such as at least 60 days' storage at 4 degrees C.

### Yeast-Produced Constructs for Oral Administration

In one aspect of the invention there is provided a construct for use in the treatment by oral administration of a disease of the intestinal tract with a first immunoglobulin chain variable domain and a second immunoglobulin chain variable domain, wherein the construct comprises the first immunoglobulin chain variable domain and the second immunoglobulin chain variable domain connected by a labile peptide linker, wherein:
(i) the labile peptide linker is labile to one or more proteases present in the intestinal tract,
(ii) the labile peptide linker is stable to yeast proteases and
(iii) the first and second immunoglobulin chain variable domains are substantially resistant to said one or more proteases and
wherein the construct is produced in yeast.

In a further aspect of the invention there is provided a method of treating by oral administration a disease of the intestinal tract with a first immunoglobulin chain variable domain and a second immunoglobulin chain variable domain from a construct, wherein the construct comprises the first immunoglobulin chain variable domain and the second immunoglobulin chain variable domain connected by a labile peptide linker, wherein:
(i) the labile peptide linker is labile to one or more proteases present in the intestinal tract,
(ii) the labile peptide linker is stable to yeast proteases and
(iii) the first and second immunoglobulin chain variable domains are substantially resistant to said one or more proteases;
wherein the construct is produced in yeast.

In a further aspect of the invention there is provided a method of delivering a first immunoglobulin chain variable domain and a second immunoglobulin chain variable domain to the intestinal tract comprising producing in yeast and then orally administering a construct comprising the first immunoglobulin chain variable domain and the second immunoglobulin chain variable domain connected by a labile peptide linker, wherein:
(i) the labile peptide linker is labile to one or more proteases present in the intestinal tract,
(ii) the labile peptide linker is stable to yeast proteases and
(iii) the first and second immunoglobulin chain variable domains are substantially resistant to said one or more proteases.

In a further aspect of the invention there is provided a method of making a construct comprising a first immunoglobulin chain variable domain and a second immunoglobulin chain variable domain connected by a labile peptide linker, wherein:
(i) the labile peptide linker is labile to one or more proteases present in the intestinal tract,
(ii) the labile peptide linker is stable to yeast proteases and
(iii) the first and second immunoglobulin chain variable domains are substantially resistant to said one or more proteases;
comprising the step of producing the construct in yeast.

Suitably the step of producing the construct in yeast is performed by providing a host yeast cell which is capable of expressing the construct of the invention, transformed with a vector, wherein the vector comprises a polynucleotide encoding the construct of the invention and wherein the host yeast cell is exposed to conditions suitable for expression of the construct of the invention.

Suitably the first immunoglobulin chain variable domain and the second immunoglobulin chain variable domain are substantially resistant to yeast proteases.

Suitably the methods outlined above further comprise the step of purifying the construct.

Suitably the labile peptide linker is cleaved in the intestinal tract by the one or more proteases present in the intestinal tract. In a further aspect of the invention, there is provided a construct obtained by any of the above methods.

The constructs for use and methods described above relate to the production of a construct in yeast. However, these constructs for use and methods are also equally applicable to production of a construct in any expression organism which produces proteases which may cleave a peptide linker, such as mammalian cells or moulds (such as moulds from any one or more of the genera Acremonium, Alternaria, Aspergillus, Chrysosporium, Cladosporium, Dictyostelium, Fusarium, Mucor, Penicillium, Rhizopus, Stachybotrys, Trichoderma and Trichophyton).

Particular characteristics of these embodiments of the invention are further discussed as follows.

1. a Labile Peptide Linker which is Labile to One or More Proteases Present in the Intestinal Tract

Examples 5 and 6 below provide details on specific constructs of the invention which comprise labile peptide linkers which are labile to one or more proteases present in the intestinal tract.

Suitably the labile peptide linker is labile to one or more proteases present in the intestinal tract such that greater than 50%, such as greater than 60%, such as greater than 70%, such as greater than 80%, such as greater than 85%, such as greater than 90% by mass of the construct is cleaved into first and second immunoglobulin chain variable domains after 160 minutes, more suitably after 105 minutes, more suitably after 60 minutes, more suitably after 25 minutes, more suitably after 10 minutes after mixing in the Trypsin Protease Assay.

2. a Labile Peptide Linker which is Stable to Yeast Proteases

Example 7 below provides details on specific constructs of the invention which comprise labile peptide linkers which are stable to yeast proteases. Example 9 below provides details on a specific construct (ID4A) which is not stable to yeast proteases.

The stability of a linker to yeast proteases may be assessed using the Yeast Expression Protocol outlined as follows.

### The Yeast Expression Protocol:

The following protocol outlines a method for the cloning of polypeptides (such as constructs comprising ICVDs or monomeric ICVDs) into the chromosome of Saccharomyces cerevisiae such that induction in a suitable growth medium results in polypeptide expression and secretion into the extracellular supernatant.

An S. cerevisiae strain of the following description is used for this process: The production strain used for expression and manufacture of polypeptides is a derivative of the CEN.PK series from the EUROSCARF collection (EUROpean Saccharomyces Cerevisiae ARchive for Functional analysis). The genotype of the CEN.PK strain is: MATa/MATα ura3-52/ura3-52; trp1-289/trp1-289; leu2-3,112/leu2-3,112; his3A 1/his3A 1; MAL2-8C/MAL2-8C; SUC2/SUC2. This strain is then further modified to inhibit the ability to grow on galactose by deletion of the galactokinase gene (gal1::URA3). This is the final strain used for transformation by the polypeptide expression constructs.

Monomeric or multimeric DNA constructs, in which polypeptides (such as constructs comprising ICVDs or monomeric ICVDs) joined by protein linkers, are cloned into a suitable multi-copy chromosomal integration vector (Lopes et al. 1991. Gene. 105, 83-90), generating an integration cassette. The integration cassette may include an inducible promoter (for example pGal7, Nogi & Fukasawa (1983). Nucleic Acids Res. 11(24):8555-68), the polypeptide encoding region, a signal sequence that encodes secretion into the extracellular supernatant in yeast (Hashimoto et al. 1998. Protein Engineering. 11 (75-77)) fused immediately upstream of the first amino acid in the polypeptide coding region, an auxotrophic selection marker and DNA sequences that contribute to recombination into the chromosome.

Transformation of competent yeast cells with linear DNA encoding the integration cassette and subsequent selection on a suitable auxotrophic medium (for example omitting leucine where a leucine biosynthesis gene is the selection marker) results in integration and amplification at the rDNA locus, such that 100-200 copies of the expression construct may be present in the cell. Following the removal of the selective pressure the expression construct remains stably integrated into the chromosome. Alternatively, polypeptide production may be achieved from a multi-copy episomal vector based on the yeast 2 μM plasmid with a similar expression cassette, without the need for chromosomal integration.

To induce polypeptide expression, a colony of the resulting yeast strain is inoculated into 5 mL of yeast soytone broth supplemented with 2% glucose and grown overnight at 30° C. (150-200 rpm). The following day 50 mL of yeast soytone broth containing 2% glucose and 0.5% galactose (for induction) in a 500 mL Erlenmeyer flask is inoculated with the entire 5 mL overnight culture. The resulting induction culture is incubated at 30° C., 200 rpm for 3 days. The culture is then spun down at 4200 rpm for 20 min in a swing rotor centrifuge to remove yeast cells. The supernatant is then filtered through 0.45 μm and 0.2 μm filters in series.

After carrying out the Yeast Expression Protocol, the polypeptide may then optionally undergo a storage period at 4° C. in the yeast supernatant under sterile conditions.

Suitably a construct of the invention is stable to yeast proteases such that no more than 10%, more suitably no more than 5%, more suitably no more than 1% by mass of the construct is cleaved into first and second immunoglobulin chain variable domains after producing the construct using the Yeast Expression Protocol (i.e. with no subsequent storage period) and then optionally storing for 2 days, more suitably 4 days or more suitably 9 days. “0 days storage” as used herein refers to no subsequent storage period.

Yeast-produced polypeptides may be distinct from those produced using alternative expression organisms such as bacteria, in that yeast-produced polypeptides may comprise post translational modifications such as glycosylation.

3. a First and Second Immunoglobulin Chain Variable Domain which are Substantially Resistant to One or More Proteases Present in the Intestinal Tract

Examples 5 and 6 below provide details on specific constructs of the invention which comprise first and second immunoglobulin chain variable domains, wherein the first and second immunoglobulin chain variable domains are substantially resistant to one or more proteases present in the intestinal tract.

Suitably the first immunoglobulin chain variable domain and the second immunoglobulin chain variable domain are substantially resistant to one or more proteases present in the intestinal tract such that at least 70%, such as at least 80%, such as at least 90%, such as at least 95%, such as at least 99%, such as about 100% by mass of the first immunoglobulin chain variable domain and at least 70%, such as at least 80%, such as at least 90%, such as at least 95%, such as at least 99%, such as about 100% by mass of the second immunoglobulin chain variable domain remain uncleaved after 10 minutes, more suitably after 25 minutes, more suitably after 60 minutes, more suitably after 105 minutes, more suitably after 160 minutes after mixing in the Trypsin Protease Assay.

4. a First and Second Immunoglobulin Chain Variable Domain which are Substantially Resistant to Yeast Proteases

Example 7 below provides details on specific constructs of the invention which comprise immunoglobulin chain variable domains, wherein the immunoglobulin chain variable domains are stable to yeast proteases.

Suitably the first immunoglobulin chain variable domain and the second immunoglobulin chain variable domain are substantially resistant to yeast proteases such that no more than 10%, more suitably no more than 5%, more suitably no more than 1% by mass of the first or second immunoglobulin chain variable domain are cleaved after producing the first or second immunoglobulin chain variable domain using the Yeast Expression Protocol and then optionally storing for 2 days, more suitably 4 days or more suitably 9 days.

### Assessment of Stability and Lability Under Points 1-4 Above

Suitably the cleaved mass of the construct and/or the uncleaved mass of the first and second immunoglobulin chain variable domains and/or the cleaved mass of the first and second immunoglobulin chain variable domains are assessed by gel electrophoresis followed by visual inspection of the gel. More suitably, assessment is by quantitative gel electrophoresis. Alternatively, assessment is by gel electrophoresis followed by mass spectrometry.

Bands on a gel corresponding to cleaved construct and uncleaved immunoglobulin chain variable domain may be identified as those bands having approximately half the mass of the construct (i.e. the mass of one constituent immunoglobulin chain variable domain), by comparison with molecular weight marker. Bands on a gel corresponding to cleaved immunoglobulin chain variable domains are identifiable by having a molecular weight lower than that of the whole constituent immunoglobulin chain variable domain, by comparison to molecular weight marker bands.

In one embodiment, the labile peptide linker of the construct of the invention does not comprise a polypeptide disclosed in WO2009/021754. More specifically, in one embodiment the labile peptide linker of the construct of the invention does not comprise the sequence GGGGSDDDDKGGGGS (SEQ ID NO: 4).

Polypeptides, antigen-binding polypeptides, antibodies and antibody fragments including immunoglobulin chain variable domains (ICVD) such as the VH and VHH Polypeptides are organic polymers consisting of a number of amino acid residues bonded together in a chain. As used herein, ‘polypeptide’ is used interchangeably with ‘protein’ and ‘peptide’. Polypeptides are said to be antigen-binding when they contain one or more stretches of amino acid residues which form an antigen-binding site, capable of binding to an epitope on a target antigen with an affinity (suitably expressed as a Kd value, a Ka value, a kₒₙ-rate and/or a k_{off}-rate, as further described herein). Antigen-binding polypeptides include polypeptides such as antibodies, antibody fragments and antigen-binding fragments. A polypeptide may comprise a region which is capable of binding a target with high affinity (suitably expressed as a Kd value, a Ka value, a kₒₙ-rate and/or a k_{off}-rate, as further described herein). Such polypeptides include DARPins (Binz et al. Journal of Molecular Biology 332(2):489-503), Affimers™, Fynomers™, Centyrins, Nanofitins® and cyclic peptides.

A conventional antibody or immunoglobulin (Ig) is a protein comprising four polypeptide chains: two heavy (H) chains and two light (L) chains. Each chain is divided into a constant region and a variable domain. The heavy chain variable domains are abbreviated herein as VHC, and the light (L) chain variable domains are abbreviated herein as VLC. These domains, domains related thereto and domains derived therefrom, are referred to herein as immunoglobulin chain variable domains. The VHC and VLC domains can be further subdivided into regions of hypervariability, termed “complementarity determining regions” (“CDRs”), interspersed with regions that are more conserved, termed “framework regions” (“FRs”). The framework and complementarity determining regions have been precisely defined (Kabat et al 1991 Sequences of Proteins of Immunological Interest, Fifth Edition U.S. Department of Health and Human Services, NIH Publication Number 91-3242, herein incorporated by reference in its entirety). In a conventional antibody, each VHC and VLC is composed of three CDRs and four FRs, arranged from amino-terminus to carboxy-terminus in the following order: FR1, CDR1, FR2, CDR2, FR3, CDR3, FR4. The conventional antibody tetramer of two heavy immunoglobulin chains and two light immunoglobulin chains is formed with the heavy and the light immunoglobulin chains inter-connected by e.g. disulfide bonds, and the heavy chains similarity connected. The heavy chain constant region includes three domains, CH1, CH2 and CH3. The light chain constant region is comprised of one domain, CL. The variable domain of the heavy chains and the variable domain of the light chains are binding domains that interact with an antigen. The constant regions of the antibodies typically mediate the binding of the antibody to host tissues or factors, including various cells of the immune system (e.g. effector cells) and the first component (C1q) of the classical complement system. The term antibody includes immunoglobulins of types IgA, IgG, IgE, IgD, IgM (as well as subtypes thereof), wherein the light chains of the immunoglobulin may be kappa or lambda types. The overall structure of immunoglobulin-gamma (IgG) antibodies assembled from two identical heavy (H)-chain and two identical light (L)-chain polypeptides is well established and highly conserved in mammals (Padlan 1994 Mol Immunol 31:169-217).

An exception to conventional antibody structure is found in sera of Camelidae. In addition to conventional antibodies, these sera possess special IgG antibodies. These IgG antibodies, known as heavy-chain antibodies (HCAbs), are devoid of the L chain polypeptide and lack the first constant domain (CH1). At its N-terminal region, the H chain of the homodimeric protein contains a dedicated immunoglobulin chain variable domain, referred to as the VHH, which serves to associate with its cognate antigen (Muyldermans 2013 Annu Rev Biochem 82:775-797, Hamers-Casterman et al 1993 Nature 363(6428):446-448, Muyldermans et al 1994 Protein Eng 7(9):1129-1135, herein incorporated by reference in their entirety).

An antigen-binding fragment (or “antibody fragment” or “immunoglobulin fragment”) as used herein refers to a portion of an antibody that specifically binds to a target (e.g. a molecule in which one or more immunoglobulin chains is not full length, but which specifically binds to a target). Examples of binding fragments encompassed within the term antigen-binding fragment include:
(i) a Fab fragment (a monovalent fragment consisting of the VLC, VHC, CL and CH1 domains);
(ii) a F(ab′)2 fragment (a bivalent fragment comprising two Fab fragments linked by a disulfide bridge at the hinge region);
(iii) a Fd fragment (consisting of the VHC and CH1 domains);
(iv) a Fv fragment (consisting of the VLC and VHC domains of a single arm of an antibody);
(v) an scFv fragment (consisting of VLC and VHC domains joined, using recombinant methods, by a synthetic linker that enables them to be made as a single protein chain in which the VLC and VHC regions pair to form monovalent molecules);
(vi) a VH (an immunoglobulin chain variable domain consisting of a VHC domain (Ward et al Nature 1989 341:544-546);
(vii) a VL (an immunoglobulin chain variable domain consisting of a VLC domain);
(viii) a V-NAR (an immunoglobulin chain variable domain consisting of a VHC domain from chondrichthyes IgNAR (Roux et al 1998 Proc Natl Acad Sci USA 95:11804-11809 and Griffiths et al 2013 Antibodies 2:66-81, herein incorporated by reference in their entirety)
(ix) a VHH.

The total number of amino acid residues in a VHH or VH may be in the region of 110-130, is suitably 112-120, and is most suitably 115.

Constructs of the invention comprising polypeptides and labile peptide linkers may for example be obtained by preparing a nucleic acid encoding two or more polypeptides and a labile peptide linker using techniques for nucleic acid synthesis, followed by expression of the nucleic acid thus obtained (as detailed further herein). According to a specific embodiment, a construct according to the invention does not have an amino acid sequence which is exactly the same as (i.e. shares 100% sequence identity with) the amino acid sequence of a naturally occurring polypeptide.

Suitably the first and/or second polypeptide of the construct of the invention is an antigen-binding polypeptide, more suitably the first and/or second antigen-binding polypeptide is an immunoglobulin chain variable domain, an antibody or an antigen-binding fragment thereof.

More suitably the antigen-binding fragment is selected from the group consisting of: VLs, V-NARs, scFvs, Fab fragments, F(ab′)2 fragments or immunoglobulin chain variable domains such as VHHs and VHs.

### Specificity, Affinity and Avidity

Specificity refers to the number of different types of antigens or antigenic determinants to which a particular antigen-binding polypeptide can bind. The specificity of an antigen-binding polypeptide is the ability of the antigen-binding polypeptide to recognise a particular antigen as a unique molecular entity and distinguish it from another.

Affinity, represented by the equilibrium constant for the dissociation of an antigen with an antigen-binding polypeptide (Kd), is a measure of the binding strength between an antigenic determinant and an antigen-binding site on an antigen-binding polypeptide: the lesser the value of the Kd, the stronger the binding strength between an antigenic determinant and the antigen-binding polypeptide (alternatively, the affinity can also be expressed as the affinity constant (Ka), which is 1/Kd). Affinity can be determined by known methods, depending on the specific antigen of interest.

Avidity is the measure of the strength of binding between an antigen-binding polypeptide and the pertinent antigen. Avidity is related to both the affinity between an antigenic determinant and its antigen-binding site on the antigen-binding polypeptide and the number of pertinent binding sites present on the antigen-binding polypeptide.

Suitably, antigen-binding polypeptides bind with a dissociation constant (Kd) of 10⁻⁶ to 10⁻¹² M, more suitably 10⁻⁷ to 10⁻¹² M, more suitably 10⁻⁸ to 10⁻¹² M and more suitably 10⁻⁹ to 10⁻¹² M.

Any Kd value less than 10⁻⁶ is considered to indicate binding. Specific binding of an antigen-binding polypeptide to an antigen or antigenic determinant can be determined in any suitable known manner, including, for example, Scatchard analysis and/or competitive binding assays, such as radioimmunoassays (RIA), enzyme immunoassays (EIA) and sandwich competition assays, and the different variants thereof known in the art.

### Polypeptide and Polynucleotide Sequences

The construct of the invention, the labile peptide linker and the first and second polypeptides comprised within the construct all comprise amino acid residues.

Suitably the first and second polypeptides each comprise or more suitably consists of a sequence selected from the group consisting of SEQ ID NO: 18 and SEQ ID NO: 19

Suitably the construct comprises or more suitably consists of a sequence selected from the group consisting of SEQ ID NOs: 7 to 17. Suitably the construct comprises or more suitably consists of a sequence selected from the group consisting of SEQ ID NOs: 8 to 11 and 13 to 16.

Suitably the labile peptide linker comprises or more suitably consists of a sequence selected from the group consisting of SEQ ID NOs: 1 to 6. Suitably the labile peptide linker comprises or more suitably consists of a sequence selected from the group consisting of SEQ ID NOs: 2 to 5.

An amino acid residue in regions of the construct other than the labile peptide linker can be replaced with another amino acid residue of similar chemical structure and which is expected to have little influence on the function, activity or other biological properties of the polypeptide. Such a substitution is a “conservative” substitution Such conservative substitutions suitably are substitutions in which one amino acid within the following groups is substituted by another amino acid residue from within the same group:

| Group | Amino acid residue |
|---|---|
| Non-polar aliphatic | Glycine |
| | Alanine |
| | Valine |
| | Leucine |
| | Isoleucine |
| Aromatic | Phenylalanine |
| | Tyrosine |
| | Tryptophan |
| Polar uncharged | Serine |
| | Threonine |
| | Asparagine |
| | Glutamine |
| Negatively charged | Aspartate |
| | Glutamate |
| Positively charged | Lysine |
| | Arginine |

The construct, excluding the labile peptide linker, may suitably include one or more conservative substitutions.

In one aspect of the invention there is provided a nucleic acid encoding the construct of the invention.

For the avoidance of doubt, the single-letter amino acid code is as follows:
G—Glycine (Gly), P—Proline (Pro), A—Alanine (Ala), V—Valine (Val), L—Leucine (Leu), I—Isoleucine (Ile), M—Methionine (Met), C—Cysteine (Cys), F—Phenylalanine (Phe), Y—Tyrosine (Tyr), W—Tryptophan (Trp), H—Histidine (His), K—Lysine (Lys), R—Arginine (Arg), Q—Glutamine (Gln), N—Asparagine (Asn), E—Glutamic Acid (Glu), D—Aspartic Acid (Asp), S—Serine (Ser), T—Threonine (Thr).

### Multimers

A construct according to the invention comprises a first polypeptide and a second polypeptide. The inventive construct is therefore multimeric and may suitably be multivalent. Such a construct may comprise a first polypeptide and a second polypeptide which are identical. A construct consisting of two identical polypeptides is a “homobihead”. In one aspect of the invention there is provided a construct comprising two identical polypeptides. Alternatively, a construct may consist of a first polypeptide and a second polypeptide which are different from one another (a “heterobihead”).

Constructs can be multivalent and/or multispecific. A multivalent construct (such as a bivalent construct) comprises two or more binding polypeptides and therefore provides two or more sites at which attachment to antigens can occur suitably before or after cleavage of the labile peptide linker. An example of a multivalent construct could be a homobihead or a heterobihead. A multispecific construct such as a bispecific construct comprises two different binding polypeptides which present two sites at which either (a) attachment to two different antigens can occur or (b) attachment to two different epitopes on the same antigen can occur, suitably before or after cleavage of the labile peptide linker. An example of a multispecific construct could be a heterobihead. A multispecific construct is multivalent.

A construct of the invention may comprise an additional third polypeptide (connected to the first polypeptide by a peptide linker) and may also comprise or consist of an additional fourth polypeptide (connected to the second polypeptide by a peptide linker), wherein the third and fourth polypeptides are substantially resistant to the one or more proteases present in the intestinal tract and are as defined herein in respect of the first and second polypeptides. A construct of the invention consisting of four polypeptides is known as a ‘quadrahead’. Suitably, the peptide linkers are substantially resistant to the one or more proteases present in the intestinal tract or alternatively the peptide linkers are labile peptide linkers as defined herein.

Suitably the first, second, third and/or fourth polypeptide has a molecular weight of no greater than 300 kDa, such as 250 kDa, such as 200 kDa, such as 180 kDa, such as 160 kDa, such as 140 kDa, such as 120 kDa, such as 100 kDa, such as 80 kDa, such as 60 kDa.

### The Gastrointestinal Tract and Digestive Enzymes

The gastrointestinal tract (GIT) is an organ system responsible for consuming and digesting foodstuffs, absorbing nutrients, and expelling waste. In humans and other mammals, the GIT consists of the oesophagus, stomach, small intestine (duodenum, jejunum and ileum) and large intestine (cecum, colon, rectum and anal canal). Various pathogens may colonise and various diseases may manifest in different areas of the GIT. The intestinal tract (as opposed to the gastrointestinal tract) consists of the small and large intestine.

The different parts of the gastrointestinal tract each contain a complex mixture of digestive enzymes. These digestive enzymes include proteases, lipases, amylases and nucleases. Proteases include serine proteases, threonine proteases, cysteine proteases, aspartate proteases, glutamic acid proteases and metalloproteases. Proteases are involved in digesting polypeptide chains into shorter fragments by splitting the peptide bonds that link amino acid residues (proteolysis). Some detach the terminal amino acids from the protein chain (exopeptidases), others attack internal peptide bonds of a protein (endopeptidases).

Proteolysis can be highly promiscuous such that a wide range of protein substrates are hydrolysed. This is the case for proteases which cleave the wide array of ingested polypeptides in the intestinal tract into smaller polypeptide fragments.

Many proteases typically bind to a single amino acid (a labile site) on the substrate and so only have specificity for that residue. The proteases present in the intestinal tract include trypsin, trypsin-like proteases, chymotrypsin, chymotrypsin-like proteases, carboxypeptidase, elastase, aminopeptidase, carboxypeptidase and enteropeptidase. Trypsin-like proteases cleave peptide bonds following lysine or arginine residues. Chymotrypsin-like proteases cleave peptide bonds following hydrophobic residues, such as tyrosine, phenylalanine, tryptophan, leucine and methionine. Particularly tyrosine, phenylalanine and tryptophan.

Suitably the labile peptide linker is labile to one or more proteases present in the intestinal tract and wherein the first and second polypeptides are substantially resistant to said one or more proteases, wherein said one or more proteases are present in the small or large intestine, more suitably the jejunum, the ileum and/or the cecum. Suitably the one or more proteases are serine proteases. Suitably the one or more proteases are selected from the group consisting of enteropeptidase, trypsin, trypsin-like proteases, chymotrypsin and chymotrypsin-like proteases.

Suitably the first and second polypeptides of the construct of the invention are substantially resistant to all proteases present in the intestinal tract. Such proteases include proteases sourced from gastrointestinal tract commensal microflora or pathogenic bacteria, for example wherein the proteases are cell membrane-attached proteases, secreted proteases and proteases released on cell lysis. Suitably the intestinal tract is a mammalian intestinal tract, such as a human, simian, murine, bovine, ovine or porcine intestinal tract.

### Diseases of the GIT

Diseases of the GIT refer to diseases involving the gastrointestinal tract, namely the oesophagus, stomach, small intestine (duodenum, jejunum and ileum) and large intestine (cecum, colon, rectum and anal canal). The construct of the invention may be used in the treatment or prevention of such diseases. Exemplary diseases of the GIT are described below.

Autoimmune Diseases and/or Inflammatory Diseases of the GIT

Autoimmune diseases develop when the immune system responds adversely to normal body tissues. Autoimmune disorders may result in damage to body tissues, abnormal organ growth and/or changes in organ function. The disorder may affect only one organ or tissue type or may affect multiple organs and tissues. Organs and tissues commonly affected by autoimmune disorders include blood components such as red blood cells, blood vessels, connective tissues, endocrine glands such as the thyroid or pancreas, muscles, joints and skin. An inflammatory disease is a disease characterised by inflammation. Many inflammatory diseases are autoimmune diseases and vice-versa.

The chronic inflammatory bowel diseases (IBD) Crohn's disease and ulcerative colitis, which afflict both children and adults, are examples of autoimmune and inflammatory diseases of the GIT (Hendrickson et al 2002 Clin Microbiol Rev 15(1):79-94, herein incorporated by reference in its entirety). Ulcerative colitis is defined as a condition where the inflammatory response and morphologic changes remain confined to the colon. The rectum is involved in 95% of patients. Inflammation is largely limited to the mucosa and consists of continuous involvement of variable severity with ulceration, edema, and hemorrhage along the length of the colon (Hendrickson et al 2002 Clin. Microbiol Rev 15(1):79-94, herein incorporated by reference in its entirety). Ulcerative colitis is usually manifested by the presence of blood and mucus mixed with stool, along with lower abdominal cramping which is most severe during the passage of bowel movements. Clinically, the presence of diarrhoea with blood and mucus differentiates ulcerative colitis from irritable bowel syndrome, in which blood is absent. Unlike ulcerative colitis, the presentation of Crohn's disease is usually subtle, which leads to a later diagnosis. Factors such as the location, extent, and severity of involvement determine the extent of gastrointestinal symptoms. Patients who have ileocolonic involvement usually have postprandial abdominal pain, with tenderness in the right lower quadrant and an occasional inflammatory mass.

Suitably the pharmaceutical composition or construct of the invention is for use in the treatment of an autoimmune and/or inflammatory disease of the GIT selected from the list consisting of Crohn's disease, ulcerative colitis, irritable bowel syndrome, diabetes type II, glomerulonephritis, autoimmune hepatitis, Sjogren's syndrome, celiac disease and drug- or radiation-induced mucositis (most suitably Crohn's disease).

### Infection of the GIT

Viral, bacterial, parasitic and other pathogenic infections can occur in the GIT. These may be confined to the GIT or initiated in the GIT before spreading to other parts of the body. The construct of the invention may be used for the treatment or prevention of bacterial infection including infection by common bacterial GIT pathogens including Escherichia coli, Salmonella, Campylobacter, Vibrio cholerae, Shigella, Clostridium perfringens, Clostridium difficile, Bacillus cereus, Vibrio parahaemolyticus, Yersinia enterocolitica. The construct of the invention may be used for the treatment or prevention of viral infection including common viral GIT pathogens which include rotavirus, norovirus and small round viruses. Suitably the construct of the invention is for use in the treatment or prevention of nosocomial infection. Suitably the construct of the invention is for use in the treatment or prevention of C. difficile infection.

Suitably, the first and/or second polypeptide of the construct of the invention binds to a target accessible via the intestinal tract such as a target within the intestinal tract. Suitably the target is a deleterious agent originating from an intestinal tract resident pathogenic microbe. Suitably the target is selected from the group consisting of: an interleukin (such as IL-1, IL-1ra, IL-4, IL-5, IL-6, IL-7, IL-8, IL-9, IL-10, IL-11, IL-12, IL-13, IL-15, IL-17, IL-18 and IL-23), an interleukin receptor (such as IL-6R and IL-7R), a transcription factor (such as NF-kB), a cytokine (such as TNF-alpha, IFN-gamma TGF-beta and TSLP), a transmembrane protein (such as gp130 and CD3), a surface glycoprotein (such as CD4, CD20, CD40), a soluble protein (such as CD40L), an integrin (such as a4b7 and AlphaEbeta7), an adhesion molecule (such as MAdCAM), a chemokine (such as IP10 and CCL20), a chemokine receptor (such as CCR2 and CCR9), an inhibitory protein (SMAD7), a kinase (such as JAK3), a G protein-coupled receptor (such as sphingosine-1-P receptor) and a toxin (such as C. difficile toxin A and C. difficile toxin B); more suitably the target is selected from the group consisting of: TNF-alpha, C. difficile toxin A, C. difficile toxin B, CD3 and IL-6R; more suitably TNF-alpha or C. difficile toxin A. Suitably the target of the first and second polypeptides are identical or different.

In one embodiment the first and/or second polypeptide of the construct of the invention (such as the first and/or second immunoglobulin chain variable domain) does not bind to an interleukin (such as IL-1, IL-1ra, IL-4, IL-5, IL-6, IL-7, IL-8, IL-9, IL-10, IL-11, IL-12, IL-13, IL-15, IL-17, IL-18 and IL-23).

In a further embodiment the first and/or second polypeptide of the construct of the invention (such as the first and/or second immunoglobulin chain variable domain) does not bind to an interleukin receptor (such as IL-6R and IL-7R).

In a further embodiment the first and/or second polypeptide of the construct of the invention (such as the first and/or second immunoglobulin chain variable domain) does not bind to a transcription factor (such as NF-kB).

In a further embodiment the first and/or second polypeptide of the construct of the invention (such as the first and/or second immunoglobulin chain variable domain) does not bind to a cytokine (such as TNF-alpha, IFN-gamma TGF-beta and TSLP).

In a further embodiment the first and/or second polypeptide of the construct of the invention (such as the first and/or second immunoglobulin chain variable domain) does not bind to a transmembrane protein (such as gp130 and CD3).

In a further embodiment the first and/or second polypeptide of the construct of the invention (such as the first and/or second immunoglobulin chain variable domain) does not bind to a surface glycoprotein (such as CD4, CD20 and CD40).

In a further embodiment the first and/or second polypeptide of the construct of the invention (such as the first and/or second immunoglobulin chain variable domain) does not bind to a soluble protein (such as CD40L).

In a further embodiment the first and/or second polypeptide of the construct of the invention (such as the first and/or second immunoglobulin chain variable domain) does not bind to an integrin (such as a4b7 and AlphaEbeta7).

In a further embodiment the first and/or second polypeptide of the construct of the invention (such as the first and/or second immunoglobulin chain variable domain) does not bind to an adhesion molecule (such as MAdCAM) In a further embodiment the first and/or second polypeptide of the construct of the invention (such as the first and/or second immunoglobulin chain variable domain) does not bind to a chemokine (such as IP10 and CCL20).

In a further embodiment the first and/or second polypeptide of the construct of the invention (such as the first and/or second immunoglobulin chain variable domain) does not bind to a chemokine receptor (such as CCR2 and CCR9).

In a further embodiment the first and/or second polypeptide of the construct of the invention (such as the first and/or second immunoglobulin chain variable domain) does not bind to an inhibitory protein (SMAD7).

In a further embodiment the first and/or second polypeptide of the construct of the invention (such as the first and/or second immunoglobulin chain variable domain) does not bind to a kinase (such as JAK3).

In a further embodiment the first and/or second polypeptide of the construct of the invention (such as the first and/or second immunoglobulin chain variable domain) does not bind to a G protein-coupled receptor (such as sphingosine-1-P receptor).

In a further embodiment the first and/or second polypeptide of the construct of the invention (such as the first and/or second immunoglobulin chain variable domain) does not bind to a toxin (such as C. difficile toxin A and C. difficile toxin B).

### Therapeutic Use and Delivery

Suitably the construct or composition of the invention is for use as a medicament, suitably administered by oral administration, suitably for use in the treatment or prevention of diseases of the GIT (see intra). The construct or composition of the invention may also be used in the treatment or prevention of other medical conditions by oral administration such as metabolic disorders, such as obesity. In one embodiment, the construct or composition of the invention is intended to have local effect in the intestinal tract. In one embodiment, the construct or composition of the invention is not for use in the treatment or prevention of diseases by delivery into the circulation in therapeutically effective quantities.

In one aspect of the invention there is provided a method of treating autoimmune disease or C. difficile infection comprising administering to a person in need thereof a therapeutically effective amount of the inventive construct or composition.

A therapeutically effective amount of a construct of the invention is an amount which is effective, upon single or multiple dose administration to a subject, in neutralising the biological effects of a chosen target to a significant extent in a subject. A therapeutically effective amount may vary according to factors such as the disease state, age, sex, and weight of the individual, and the ability of the construct to elicit a desired response in the individual. A therapeutically effective amount is also one in which any toxic or detrimental effects of the construct are outweighed by the therapeutically beneficial effects. The construct of the invention can be incorporated into pharmaceutical compositions suitable for oral administration to a subject. The construct of the invention can be in the form of a pharmaceutically acceptable salt.

In one aspect of the invention there is provided a method of treating a disease with a monomeric antibody or a monomeric antigen binding fragment thereof, comprising administering to a subject the inventive construct. There is also provided a method of treating a disease with two or more monomeric antibodies or monomeric antigen binding fragments thereof, comprising administering to a subject the inventive construct.

A pharmaceutical composition of the invention is formulated for oral delivery. The pharmaceutical compositions of the invention may be in a variety of forms. These include, for example, liquid, semi-solid and solid dosage forms, such as liquid solutions, dispersions or suspensions, tablets, pills and powders. Solid dosage forms are preferred. The pharmaceutical composition may comprise a pharmaceutically acceptable excipient, and suitably may be used in the form of ingestible tablets, buccal tablets, troches, capsules, elixirs, suspensions, syrups, wafers, and the like.

Typically, the pharmaceutical composition comprises a construct of the invention and a pharmaceutically acceptable excipient such as a carrier. Examples of pharmaceutically acceptable carriers include one or more of water, saline, phosphate buffered saline, dextrose, glycerol, ethanol and the like, as well as combinations thereof. Pharmaceutically acceptable carriers may further comprise minor amounts of auxiliary substances such as wetting or emulsifying agents, preservatives or buffers, which enhance the shelf life or effectiveness of the polypeptide or construct of the invention. Pharmaceutical compositions may include antiadherents, binders, coatings, disintegrants, flavours, colours, lubricants, sorbents, preservatives, sweeteners, freeze dry excipients (including lyoprotectants) or compression aids. Suitably, the construct of the invention is lyophilised before being incorporated into a pharmaceutical composition.

Suitably, the first and second polypeptides of the construct are substantially resistant to all proteases present in the intestinal tract by virtue of the inherent properties of the polypeptides or construct itself.

A polypeptide of the invention may also be provided with an enteric coating. An enteric coating is a polymer barrier applied on oral medication which protects the polypeptide from the low pH of the stomach. Materials used for enteric coatings include fatty acids, waxes, shellac, plastics, and plant fibers. Suitable enteric coating components include methyl acrylate-methacrylic acid copolymers, cellulose acetate succinate, hydroxy propyl methyl cellulose phthalate, hydroxy propyl methyl cellulose acetate succinate (hypromellose acetate succinate), polyvinyl acetate phthalate (PVAP), methyl methacrylate-methacrylic acid copolymers, sodium alginate and stearic acid. Suitable enteric coatings include pH-dependent release polymers. These are polymers which are insoluble at the highly acidic pH found in the stomach, but which dissolve rapidly at a less acidic pH. Thus, suitably, the enteric coating will not dissolve in the acidic juices of the stomach (pH ˜3), but will do so in the higher pH environment present in the small intestine (pH above 6) or in the colon (pH above 7.0). The pH-dependent release polymer is selected such that the construct of the invention will be released at about the time that the dosage reaches the target region of the intestinal tract.

The pharmaceutical composition or construct of the invention may be formulated in a buffer, in order to stabilise the pH of the composition, at a concentration between 5-50, or more suitably 15-40 or more suitably 25-30 g/litre. Examples of suitable buffer components include physiological salts such as sodium citrate and/or citric acid. Suitably buffers contain 100-200, more suitably 125-175 mM physiological salts such as sodium chloride. Suitably the buffer is selected to have a pKa close to the pH of the composition or the physiological pH of the patient.

Exemplary construct concentrations in a pharmaceutical composition may range from about 1 mg/mL to about 200 mg/ml or from about 50 mg/mL to about 200 mg/mL, or from about 150 mg/mL to about 200 mg/mL.

An aqueous formulation of the construct or pharmaceutical composition of the invention may be prepared in a pH-buffered solution, e.g., at pH ranging from about 4.0 to about 7.0, or from about 5.0 to about 6.0, or alternatively about 5.5. Examples of suitable buffers include phosphate-, histidine-, citrate-, succinate-, acetate-buffers and other organic acid buffers. The buffer concentration can be from about 1 mM to about 100 mM, or from about 5 mM to about 50 mM, depending, for example, on the buffer and the desired tonicity of the formulation.

The tonicity of the pharmaceutical composition may be altered by including a tonicity modifier. Such tonicity modifiers can be charged or uncharged chemical species. Typical uncharged tonicity modifiers include sugars or sugar alcohols or other polyols, preferably trehalose, sucrose, mannitol, glycerol, 1,2-propanediol, raffinose, sorbitol or lactitol (especially trehalose, mannitol, glycerol or 1,2-propanediol). Typical charged tonicity modifiers include salts such as a combination of sodium, potassium or calcium ions, with chloride, sulfate, carbonate, sulfite, nitrate, lactate, succinate, acetate or maleate ions (especially sodium chloride or sodium sulphate); or amino acids such as arginine or histidine. Suitably, the aqueous formulation is isotonic, although hypertonic or hypotonic solutions may be suitable. The term “isotonic” denotes a solution having the same tonicity as some other solution with which it is compared, such as physiological salt solution or serum. Tonicity agents may be used in an amount of about 5 mM to about 350 mM, e.g., in an amount of 1 mM to 500 nM. Suitably, at least one isotonic agent is included in the composition.

A surfactant may also be added to the pharmaceutical composition to reduce aggregation of the formulated construct and/or minimize the formation of particulates in the formulation and/or reduce adsorption. Exemplary surfactants include polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), and sodium dodecyl sulfate (SDS). Examples of suitable polyoxyethylenesorbitan-fatty acid esters are polysorbate 20, and polysorbate 80. Exemplary concentrations of surfactant may range from about 0.001% to about 10% w/v.

A lyoprotectant may also be added in order to protect the construct of the invention against destabilizing conditions during the lyophilization process. For example, known lyoprotectants include sugars (including glucose, sucrose, mannose and trehalose); polyols (including mannitol, sorbitol and glycerol); and amino acids (including alanine, glycine and glutamic acid). Lyoprotectants can be included in an amount of about 10 mM to 500 mM.

The dosage ranges for administration of the pharmaceutical composition or construct of the invention are those to produce the desired therapeutic effect. The dosage range required depends on the precise nature of the pharmaceutical composition or construct, the target region of the intestinal tract, the nature of the formulation, the age of the patient, the nature, extent or severity of the patient's condition, contraindications, if any, and the judgement of the attending physician. Variations in these dosage levels can be adjusted using standard empirical routines for optimisation.

Suitable daily dosages of pharmaceutical composition or construct of the invention are in the range of 50 ng-50 mg per kg, such as 50 ug-40 mg per kg, such as 5-30 mg per kg of body weight. The unit dosage can vary from less than 100 mg, but typically will be in the region of 250-2000 mg per dose, which may be administered daily or more frequently, for example 2, 3 or 4 times per day or less frequently for example every other day or once per week.

Treatment of diseases also embraces treatment of exacerbations thereof and also embraces treatment of patients in remission from disease symptoms to prevent relapse of disease symptoms.

### Combination Therapy

A pharmaceutical composition of the invention may also comprise one or more active agents (e.g. active agents suitable for treating diseases such as those mentioned herein). It is within the scope of the invention to use the pharmaceutical composition of the invention in therapeutic methods for the treatment of bacterial infection, autoimmune and/or inflammatory diseases as an adjunct to, or in conjunction with, other established therapies normally used in the treatment of bacterial, autoimmune and/or inflammatory diseases.

For the treatment of irritable bowel disease (such as Crohn's disease or ulcerative colitis), possible combinations include combinations with, for example, one or more active agents selected from the list comprising: 5-aminosalicylic acid, or a prodrug thereof (such as sulfasalazine, olsalazine or bisalazide); corticosteroids (e.g. prednisolone, methylprednisolone, or budesonide); immunosuppressants (e.g. cyclosporin, tacrolimus, methotrexate, azathioprine or 6-mercaptopurine); anti-TNF-alpha antibodies (e.g., infliximab, adalimumab, certolizumab pegol or golimumab); anti-IL12/IL23 antibodies (e.g., ustekinumab); anti-IL6R antibodies or small molecule IL12/IL23 inhibitors (e.g., apilimod); Anti-alpha-4-beta-7 antibodies (e.g., vedolizumab); MAdCAM-1 blockers (e.g., PF-00547659); antibodies against the cell adhesion molecule alpha-4-integrin (e.g., natalizumab); antibodies against the IL2 receptor alpha subunit (e.g., daclizumab or basiliximab); JAK3 inhibitors (e.g., tofacitinib or R348); Syk inhibitors and prodrugs thereof (e.g., fostamatinib and R-406); Phosphodiesterase-4 inhibitors (e.g., tetomilast); HMPL-004; probiotics; Dersalazine; semapimod/CPSI-2364; and protein kinase C inhibitors (e.g. AEB-071). The most suitable combination agents are infliximab, adalimumab, certolizumab pegol or golimumab.

For the treatment of bacterial infections, such as Clostridium difficile infection, possible combinations include combinations with, for example, one or more active agents selected from the list comprising C. difficile toxoid vaccine, ampicillin, amoxicillin, vancomycin, metronidazole, fidaxomicin, linezolid, nitazoxanide, rifaximin, ramoplanin, difimicin, clindamycin, cephalosporins (such as second and third generation cephalosporins), fluoroquinolones (such as gatifloxacin or moxifloxacin), macrolides (such as erythromycin, clarithromycin, azithromycin), penicillins, aminoglycosides, trimethoprim-sulfamethoxazole, chloramphenicol, tetracycline, imipenem, meropenem, antibacterial agents, bactericides, or bacteriostats. Possible combinations also include combinations with one or more active agents which are probiotics, for example Saccharomyces boulardii or Lactobacillus rhamnosus GG.

Hence another aspect of the invention provides a pharmaceutical composition of the invention in combination with one or more further active agents, for example one or more active agents described above. In a further aspect of the invention, the pharmaceutical composition or construct is administered sequentially, simultaneously or separately with at least one active agent selected from the list above.

Similarly, another aspect of the invention provides a combination product comprising:
(A) a pharmaceutical composition or construct of the present invention; and
(B) one or more other active agents,
wherein each of components (A) and (B) is formulated in admixture with a pharmaceutically-acceptable adjuvant, diluent or carrier. In this aspect of the invention, the combination product may be either a single (combination) formulation or a kit-of-parts. Thus, this aspect of the invention encompasses a combination formulation including a pharmaceutical composition or construct of the present invention and another therapeutic agent, in admixture with a pharmaceutically acceptable adjuvant, diluent or carrier.

The invention also encompasses a kit of parts comprising components:
(i) a pharmaceutical composition or construct of the present invention in admixture with a pharmaceutically acceptable adjuvant, diluent or carrier; and
(ii) a formulation including one or more other active agents, in admixture with a pharmaceutically-acceptable adjuvant, diluent or carrier, which components (i) and (ii) are each provided in a form that is suitable for administration in conjunction with the other.

Component (i) of the kit of parts is thus component (A) above in admixture with a pharmaceutically acceptable adjuvant, diluent or carrier. Similarly, component (ii) is component (B) above in admixture with a pharmaceutically acceptable adjuvant, diluent or carrier. The one or more other active agents (i.e. component (B) above) may be, for example, any of the agents mentioned above in connection with the treatment of bacterial infection such as Clostridium difficile infection, autoimmune and/or inflammatory diseases such as IBD (e.g. Crohn's disease and/or ulcerative colitis). If component (B) is more than one further active agent, these further active agents can be formulated with each other or formulated with component (A) or they may be formulated separately. In one embodiment component (B) is one other therapeutic agent. In another embodiment component (B) is two other therapeutic agents. The combination product (either a combined preparation or kit-of-parts) of this aspect of the invention may be used in the treatment or prevention of an autoimmune disease (e.g. the autoimmune diseases mentioned herein).

### Vectors and Hosts

The term “vector”, as used herein, is intended to refer to a nucleic acid molecule capable of transporting another nucleic acid to which it has been linked. One type of vector is a plasmid, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian and yeast vectors). Other vectors (e.g. non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and vector” may be used interchangeably as the plasmid is the most commonly used form of vector. However, the invention is intended to include such other forms of expression vectors, such as viral vectors (e.g. replication defective retroviruses. adenoviruses and adeno-associated viruses), which serve equivalent functions, and also bacteriophage and phagemid systems. The invention also relates to nucleotide sequences that encode constructs of the invention. The term “recombinant host cell” (or simply “host cell”), as used herein, is intended to refer to a cell into which a recombinant expression vector has been introduced. Such terms are intended to refer not only to the particular subject cell but to the progeny of such a cell.

In one aspect of the invention there is provided a vector comprising the polynucleotide encoding the construct of the invention or cDNA comprising said polynucleotide. In a further aspect of the invention there is provided a host cell transformed with said vector, which is capable of expressing the construct of the invention. Suitably the host cell is a yeast such as a yeast belonging to the genera Aspergillus, Saccharomyces, Kluyveromyces, Hansenula or Pichia, such as Saccharomyces cerevisiae, Escherchia coli or Pichia pastoris.

It is particularly advantageous for production and convenience purposes if the labile peptide linker is cleaved by proteases present in the intestinal tract, but if the labile peptide linker is also substantially resistant to proteases of the host organism in which the construct is produced. Therefore, in one embodiment of the invention, the labile peptide linker is substantially resistant to proteases of the host organism in which the construct is produced.

### Preparative Methods

Constructs of the invention can be obtained and manipulated using the techniques disclosed for example in Green and Sambrook 2012 Molecular Cloning: A Laboratory Manual 4^{th} Edition Cold Spring Harbour Laboratory Press.

In particular, artificial gene synthesis may be used to produce a construct according to the invention (Nambiar et al 1984 Science 223:1299-1301, Sakamar and Khorana 1988 Nucl. Acids Res 14:6361-6372, Wells et al 1985 Gene 34:315-323 and Grundstrom et al 1985 Nucl. Acids Res 13:3305-3316, herein incorporated by reference in their entirety). A gene encoding a construct of the invention can be synthetically produced by, for example, solid-phase DNA synthesis. Entire genes may be synthesized de novo, without the need for precursor template DNA. To obtain the desired oligonucleotide, the building blocks are sequentially coupled to the growing oligonucleotide chain in the order required by the sequence of the product. Upon the completion of the chain assembly, the product is released from the solid phase to solution, deprotected, and collected. Products can be isolated by high-performance liquid chromatography (HPLC) to obtain the desired oligonucleotides in high purity (Verma and Eckstein 1998 Annu Rev Biochem 67:99-134).

The constructs of the invention may be fused genetically at the DNA level i.e. a polynucleotide construct which encodes the complete polypeptide construct comprising one or more polypeptides such as antigen-binding polypeptides. One way of joining multiple polypeptides via the genetic route is by linking the polypeptide coding sequences via a labile peptide linker coding sequence. For example, the carboxy-terminal end of the first polypeptide may be linked to the amino-terminal end of the next polypeptide via a labile peptide linker coding sequence. This linking mode can be extended in order to link polypeptides for the construction of tri-, tetra-, etc. functional constructs. A method for producing multivalent (such as bivalent) VHH polypeptide constructs is disclosed in WO96/34103 (herein incorporated by reference in its entirety).

Mutations can be made to the DNA or cDNA that encode constructs which are silent as to the amino acid sequence of the polypeptide, but which provide preferred codons for translation in a particular host. The preferred codons for translation of a nucleic acid in, e.g., E. coli and S. cerevisiae, are known.

Mutation of constructs can be achieved for example by substitutions, additions or deletions to a nucleic acid encoding the construct. The substitutions, additions or deletions to a nucleic acid encoding the construct can be introduced by many methods, including for example error-prone PCR, shuffling, oligonucleotide-directed mutagenesis, assembly PCR, PCR mutagenesis, in vivo mutagenesis, cassette mutagenesis, recursive ensemble mutagenesis, exponential ensemble mutagenesis, site-specific mutagenesis (Ling et al 1997 Anal Biochem 254(2):157-178, herein incorporated by reference in its entirety), gene reassembly, Gene Site Saturation Mutagenesis (GSSM), synthetic ligation reassembly (SLR) or a combination of these methods.

The modifications, additions or deletions to a nucleic acid can also be introduced by a method comprising recombination, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, ensemble mutagenesis, chimeric nucleic acid multimer creation, or a combination thereof.

Expression of constructs comprising immunoglobulin chain variable domains such as VHs and VHHs can be achieved using a suitable expression vector such as a prokaryotic cell such as bacteria, for example E. coli (for example according to the protocols disclosed in WO94/04678 and WO96/34103, which are incorporated herein by reference). Expression of immunoglobulin chain variable domains such as VHs and VHHs can also be achieved using eukaryotic cells, for example insect cells, CHO cells, Vero cells or suitably yeast cells such as yeasts belonging to the genera Aspergillus, Saccharomyces, Kluyveromyces, Hansenula or Pichia. Suitably S. cerevisiae is used (for example according to the protocols disclosed in WO94/025591, which is incorporated herein by reference).

Suitably, the construct of the invention can be produced in a fungus such as a yeast (for example, S. cerevisiae) comprising growth of the fungus on a medium comprising a carbon source wherein 50-100 wt % of said carbon source is ethanol, according to the methods disclosed in WO02/48382.

In one aspect of the invention there is provided a process for the preparation of the construct of the invention comprising the following steps:
i) cloning into a vector, such as a plasmid, the polynucleotide of the invention,
ii) transforming a cell, such as a bacterial cell or a yeast cell capable of producing the construct of the invention, with said vector in conditions allowing the production of the construct,
iii) recovering the construct, such as by affinity chromatography.

### Clauses

A set of clauses defining the invention and its preferred aspects is as follows:
1. A construct suitable for oral administration comprising a first polypeptide and a second polypeptide connected by a labile peptide linker, wherein the labile peptide linker is labile to one or more proteases present in the intestinal tract and wherein the first and second polypeptides are substantially resistant to said one or more proteases.
2. The construct according to clause 1, wherein the first polypeptide is an antigen-binding polypeptide.
3. The construct according to clause 2, wherein the antigen-binding polypeptide is an antibody or an antigen-binding fragment thereof.
4. The construct according to any one of clauses 1 to 3, wherein the second polypeptide is an antigen-binding polypeptide.
5. The construct according to clause 4, wherein the second antigen-binding polypeptide is an antibody or an antigen-binding fragment thereof.
6. The construct according to any one of clauses 1 to 5 wherein the first and second polypeptides are substantially resistant to all proteases present in the intestinal tract.
7. The construct according to any one of clauses 1 to 6 wherein the intestinal tract is a mammalian intestinal tract, such as a human, simian, murine, bovine, ovine or porcine intestinal tract.
8. The construct according to any one of clauses 1 to 7, wherein the one or more proteases are present in the small or large intestine.
9. The construct according to clause 8, wherein the one or more proteases are present in the jejunum, the ileum and/or the cecum.
10. The construct according to either clause 8 or 9, wherein the one or more proteases are serine proteases.
11. The construct according to any one of clauses 1 to 10, wherein the labile peptide linker is substantially resistant to proteases of the host organism in which the construct is produced.
12. The construct according to any one of clauses 1 to 11, wherein the labile peptide linker comprises a cleavage site for trypsin or a trypsin-like protease.
13. The construct according to clause 12, wherein the labile peptide linker comprises at least 1, such as at least 2, such as at least 3, such as at least 4, such as at least 5, such as at least 6, such as at least 7, such as at least 8, such as at least 9, such as at least 10 K residues.
14. The construct according to any one of clauses 1 to 13, wherein the labile peptide linker comprises at least 1, such as at least 2, such as at least 3, such as at least 4, such as at least 5, such as at least 6, such as at least 7, such as at least 8, such as at least 9, such as at least 10 R residues.
15. The construct according to any one of clauses 1 to 14, wherein the labile peptide linker does not comprise any P residues.
16. The construct according to any one of clauses 1 to 15, wherein all K or R residues comprised within the labile peptide linker have at least 1, such as at least 2, such as at least 3, such as at least 4, such as at least 5 shielding residues on their N-terminal side, wherein the shielding residues are selected from the list consisting of: D and E.
17. The construct according to any one of clauses 1 to 16, wherein all K or R residues comprised within the labile peptide linker have at least 1, such as at least 2, such as at least 3, such as at least 4, such as at least 5 shielding residues on their C-terminal side, wherein the shielding residues are selected from the list consisting of: D and E.
18. The construct according to either clause 16 or 17, wherein all K and R residues have at least one shielding residue adjacent to them.
19. The construct according to any one of clauses 1 to 18, wherein the labile peptide linker does not comprise any D or E residues.
20. The construct according to any one of clauses 1 to 18, wherein the labile peptide linker consists of residues selected from the list consisting of C, A, S, N, G, L, I, V, T, M, F, Y, H, K, R, W and Q.
21. The construct according to clause 20, wherein the labile peptide linker consists of residues selected from the list consisting of A, G, L, I, V, M, S, T, K and R residues.
22. The construct according to clause 21, wherein the labile peptide linker consists of residues selected from the list consisting of S, G, K and R.
23. The construct according to any one of clauses 1 to 15, wherein all K or R residues comprised within the labile peptide linker have at least 1, such as at least 2, such as at least 3, such as at least 4, such as at least 5 non-shielding residues on their N-terminal side, wherein the non-shielding residues are selected from the list consisting of: C, A, S, N, G, L, I, V, T, M, F, Y, H, W and Q.
24. The construct according to any one of clauses 1 to 15 and 23, wherein all K or R residues comprised within the labile peptide linker have at least 1, such as at least 2, such as at least 3, such as at least 4, such as at least 5 non-shielding residues on their C-terminal side, wherein the non-shielding residues are selected from the list consisting of: C, A, S, N, G, L, I, V, T, M, F, Y, H, Wand Q.
25. The construct according to either clause 23 or 24, wherein all K and R residues have at least one non-shielding residue adjacent to them.
26. The construct according to any one of clauses 23 to 25, wherein the non-shielding residues are selected from the list consisting of: A, G, L, I, V, M, S and T.
27. The construct according to clause 26, wherein the non-shielding residues are selected from the list consisting of: A, G, L, I, V and S.
28. The construct according to clause 27, wherein the non-shielding residues are selected from the list consisting of: G and S.
29. The construct according to any one of clauses 1 to 11, wherein the labile peptide linker comprises a cleavage site for chymotrypsin or a chymotrypsin-like protease.
30. The construct according to clause 29, wherein the labile peptide linker comprises at least 1, such as at least 2, such as at least 3, such as at least 4, such as at least 5, such as at least 6, such as at least 7, such as at least 8, such as at least 9, such as at least 10 residues selected from the list consisting of W, F, Y, L and M.
31. The construct according to clause 30, wherein the labile peptide linker comprises at least 1, such as at least 2, such as at least 3, such as at least 4, such as at least 5, such as at least 6, such as at least 7, such as at least 8, such as at least 9, such as at least 10 residues selected from the list consisting of W, F and Y.
32. The construct according to any one of clauses 29 to 31, wherein the labile peptide linker consists of residues selected from the list consisting of S, G, W, F, Y, L and M; such as S, G, W, F and Y.
33. The construct according to any one of clauses 1 to 32, wherein the labile peptide linker has a length of at least 3 residues, such as at least 5 residues, such as at least 10 residues.
34. The construct according to any one of clauses 1 to 33, wherein the labile peptide linker has a length of no greater than 40 residues, such as no greater than 25 residues, such as no greater than 15 residues.
35. The construct according to clause 1, wherein the labile peptide linker comprises or more suitably consists of a polypeptide sequence of the format:

[—(GₐS)ₓ—U_{c}—B—U′_{d}—(G_{b}S)_{y}—]_{z}

wherein
a is 1 to 10;
b is 1 to 10;
U is D or E;
U′ is or E;
c is 0 to 7;
d is 0 to 7;
x is 1 to 10;
y is 1 to 10
z is 1 to 10 and
B is K or R.
36. The construct according to clause 35, wherein the labile peptide linker comprises or more suitably consists of a polypeptide sequence of the format:

[—(GₐS)ₓ—B—(G_{b}S)_{y}—]_{z}

wherein
a is 1 to 10;
b is 1 to 10;
x is 1 to 10;
y is 1 to 10
z is 1 to 10 and
B is K or R.
37. The construct according to clause 36, wherein the labile peptide linker comprises or more suitably consists of a polypeptide sequence of the format:

[—(G₄S)ₓ—B—(G₄S)_{y}—]_{z}

wherein
x is 1 to 10;
y is 1 to 10
z is 1 to 10 and
B is K or R.
38. The construct according to clause 1, wherein the labile peptide linker comprises or more suitably consists of a polypeptide sequence of the format:

—B—(GₐS)ₓ—B′—

wherein
a is 1 to 10;
x is 1 to 10;
B is K or R and
B′ is K or R.
39. The construct according to clause 1, wherein the labile peptide linker comprises or more suitably consists of a polypeptide sequence of the format:

—B—(GₐS)ₓ—B′—(G_{b}S)_{y}—B″—

wherein
a is 1 to 10;
b is 1 to 10;
x is 1 to 10;
y is 1 to 10;
B is K or R
B′ is K or R and
B″ is K or R.
40. The construct according to any one of clauses 35 to 39, wherein a is 2 to 5.
41. The construct according to clause 40, wherein a is 4.
42. The construct according to any one of clauses 35 to 41, wherein b is 2 to 5.
43. The construct according to clause 42, wherein b is 4.
44. The construct according to any one of clauses 35 to 43, wherein x is 1 to 5.
45. The construct according to clause 44, wherein x is 2.
46. The construct according to clause 44, wherein x is 1.
47. The construct according to any one of clauses 35 to 46, wherein y is 1 to 5.
48. The construct according to clause 47, wherein y is 1.
49. The construct according to clause 47, wherein y is 2.
50. The construct according to any one of clauses 35 to 49, wherein z is 1 to 3.
51. The construct according to clause 50, wherein z is 1.
52. The construct according to any one of clauses 35 to 51, wherein B is K.
53. The construct according to any one of clauses 35 to 52, wherein U is D.
54. The construct according to any one of clauses 35 to 53, wherein U′ is D.
55. The construct according to any one of clauses 35 to 54, wherein c is 1.
56. The construct according to any one of clauses 35 to 55, wherein d is 1.
57. The construct according to any one of clauses 35 to 54, wherein c is 0.
58. The construct according to any one of clauses 35 to 55 or 57, wherein d is 0.
59. The construct according to any one of clauses 35 to 54, wherein c is 4 and d is 0.
60. The construct according to any one of clauses 35 to 59, wherein B′ is K.
61. The construct according to any one of clauses 35 to 60, wherein B″ is K.
62. The construct according to clause 1, wherein the labile peptide linker comprises or more suitably consists of a polypeptide sequence of the format:

[—(GₐS)ₓ—J—(G_{b}S)_{y}—]_{z}

wherein
a is 1 to 10;
b is 1 to 10;
x is 1 to 10;
y is 1 to 10
z is 1 to 10 and
J is W, F, Y, L or M.
63. The construct according to clause 62, wherein the labile peptide linker comprises or more suitably consists of a polypeptide sequence of the format:

[—(G₄S)ₓ—J—(G₄S)_{y}—]_{z}

wherein
x is 1 to 10;
y is 1 to 10
z is 1 to 10 and
J is W, F, Y, L or M.
64. The construct according to clause 1, wherein the labile peptide linker comprises or more suitably consists of a polypeptide sequence of the format:

—J—(GₐS)ₓ—J′—

wherein
a is 1 to 10;
x is 1 to 10;
J is W, F, Y, L or M and
J′ is W, F, Y, L or M.
65. The construct according to clause 1, wherein the labile peptide linker comprises or more suitably consists of a polypeptide sequence of the format:

—J—(GₐS)ₓ—J′—(G_{b}S)_{y}—J″—

wherein
a is 1 to 10;
b is 1 to 10;
x is 1 to 10;
y is 1 to 10;
J is W, F, Y, L or M
J′ is W, F, Y, L or M and
J″ is W, F, Y, L or M.
66. The construct according to any one of clauses 62 to 65, wherein a is 2 to 5.
67. The construct according to clause 66, wherein a is 4.
68. The construct according to any one of clauses 62 to 67, wherein b is 2 to 5.
69. The construct according to clause 68, wherein b is 4.
70. The construct according to any one of clauses 62 to 69, wherein x is 1 to 5.
71. The construct according to clause 70, wherein x is 2.
72. The construct according to clause 70, wherein x is 1.
73. The construct according to any one of clauses 62 to 72, wherein y is 1 to 5.
74. The construct according to clause 73, wherein y is 1.
75. The construct according to clause 73, wherein y is 2.
76. The construct according to any one of clauses 62 to 75, wherein z is 1 to 3.
77. The construct according to clause 76, wherein z is 1.
78. The construct according to any one of clauses 62 to 77, wherein J is W, F or Y.
79. The construct according to any one of clauses 62 to 78, wherein J′ is W, F or Y.
80. The construct according to any one of clauses 62 to 79, wherein J″ is W, F or Y.
81 The construct according to any one of clauses 1 to 80, wherein the first antibody or antigen binding fragment thereof binds to a first target accessible via the intestinal tract, such as a target within the intestinal tract.
82. The construct according to clause 81, wherein the first target is a first deleterious agent originating from an intestinal tract resident pathogenic microbe.
83. The construct according to clause 81, wherein the first target is selected from the group consisting of: TNF-alpha, C. difficile toxin A, C. difficile toxin B, CD3 or IL-6R.
84. The construct according to clause 83, wherein the first target is selected from the group consisting of: TNF-alpha or C. difficile toxin A.
85. The construct according to clause 81, wherein the second antibody or antigen binding fragment thereof binds to a second target accessible via the intestinal tract, such as a target within the intestinal tract.
86. The construct according to clause 85, wherein second target is a second deleterious agent originating from an intestinal tract resident pathogenic microbe.
87. The construct according to clause 85, wherein the second target is selected from the group consisting of: TNF-alpha, C. difficile toxin A, C. difficile toxin B, CD3 or IL-6R.
88. The construct according to clause 87, wherein the second target is selected from the group consisting of: TNF-alpha or C. difficile toxin A.
89. The construct according to any one of clauses 81 to 88, wherein the first and second targets are identical.
90. The construct according to any one of clauses 81 to 88, wherein the first and second targets are different.
91. The construct according to any one of clauses 1 to 90, wherein the antigen-binding fragments are selected from the group consisting of: VLs, V-NARs, scFvs, Fab fragments, F(ab′)2 fragments or immunoglobulin chain variable domains such as VHHs and VHs.
92. The construct according to clause 91, wherein the antigen-binding fragments are VHHs.
93. The construct according to clause 91, wherein the antigen-binding fragments are VHs.
94. The construct according to any one of clauses 1 to 93, wherein at least 20%, such as at least 40%, such as at least 60%, such as at least 80%, such as at least 90%, such as at least 100% by mass of the construct remains uncleaved after at least 10, such as at least 20, such as at least 30 minutes after mixing in the Faecal Protease Assay and/or Trypsin Protease Assay and/or Chymotrypsin Protease Assay.
95. The construct according to clause 1, wherein the first and second polypeptides each comprise a sequence selected from the group consisting of SEQ ID NO: 18 and SEQ ID NO: 19.
96. The construct according to clause 95 wherein the first and second polypeptides each consist of a sequence selected from the group consisting of SEQ ID NO: 18 and SEQ ID NO: 19.
97. The construct according to clause 1 wherein the construct comprises a sequence selected from the group consisting of SEQ ID NOs: 7 to 17.
98. The construct according to clause 97 wherein the construct consists of a sequence selected from the group consisting of SEQ ID NOs: 7 to 17.
99. The construct according to clause 1 wherein the labile peptide linker comprises a sequence selected from the group consisting of SEQ ID NOs: 1 to 6.
100. The construct according to clause 99 wherein the labile peptide linker consists of a sequence selected from the group consisting of SEQ ID NOs: 1 to 6.
101. The construct according to any one of clauses 1 to 94, wherein the construct comprises an additional third polypeptide connected by a peptide linker to the first polypeptide, wherein the third polypeptide is substantially resistant to the one or more proteases present in the intestinal tract.
102. The construct according to clause 101, wherein the construct comprises or consists of an additional fourth polypeptide connected by a peptide linker to the second polypeptide, wherein the fourth polypeptide is substantially resistant to the one or more proteases present in the intestinal tract.
103. The construct according to clauses 101 or 102, wherein the peptide linkers are substantially resistant to the one or more proteases present in the intestinal tract.
104. The construct according to clause 101 or 102, wherein the peptide linkers are labile peptide linkers.
105. The construct according to any one of clauses 1 to 104, wherein the construct is enterically-coated.
106. A pharmaceutical composition comprising a construct according to any one of clauses 1 to 105 and a pharmaceutically acceptable excipient.
107. The construct or composition according to any one of clauses 1 to 106 for use as a medicament.
108. The construct or composition according to clause 107 wherein the medicament is administered by oral administration.
109. The construct or composition according to clause 108 for use in the treatment of diseases of the GIT.
110. The construct or composition according to clause 109 for use in the treatment of autoimmune and/or inflammatory disease.
111. The construct or composition according to clause 110 for use in the treatment of inflammatory bowel disease such as Crohn's disease and/or ulcerative colitis.
112. The construct or composition according to clause 109 for use in the treatment of bacterial infection such as C. difficile infection.
113. A nucleic acid comprising a sequence encoding the construct according to any one of clauses 1 to 104.
114. A method of treating autoimmune disease comprising administering to a person in need thereof a therapeutically effective amount of a construct or composition according to any one of clauses 1 to 106.
115. A method of treating bacterial infection such as C. difficile infection comprising administering to a person in need thereof a therapeutically effective amount of a construct or composition according to any one of clauses 1 to 106.
116. A method of treating a disease with a monomeric polypeptide, comprising administering to a subject the construct or composition of any one of clauses 1 to 106.
117. A method of treating a disease with two or more monomeric polypeptides, comprising administering to a subject the construct or composition of any one of clauses 1 to 106.
118. A method of treating two or more diseases with two or more monomeric polypeptides, comprising administering to a subject the construct or composition of any one of clauses 1 to 106.
119. The method of any one of clauses 114 to 118 wherein the disease is an autoimmune disease and/or inflammatory disease or GIT infection.
120. A method of assaying the lability of a construct according to any one of clauses 1 to 104, comprising the steps of: (a) incubating the construct in a solution comprising trypsin, a solution comprising chymotrypsin, faecal supernatant, small intestinal fluid, or a solution comprising enteropeptidase, such as by performing the Trypsin Protease Assay of Example 1, the Chymotrypsin Protease Assay of Example 2 or the Faecal Protease Assay of Example 3, then (b) ascertaining the proportion of cleaved construct after one or more periods of incubation.
121. The method according to clause 120, wherein the level of lability is the percentage of the construct remaining uncleaved after at least 10, such as at least 20, such as at least 30 minutes after mixing in the Trypsin Protease Assay of Example 1, the Chymotrypsin Protease Assay of Example 2 or the Faecal Protease Assay of Example 3.
122. A method of delivering a monomeric antibody or a monomeric antigen binding fragment thereof to a targeted region of the intestinal tract, comprising the steps of: (a) performing the method according to either clauses 120 or 121 then (b) selecting a construct with an appropriate level of lability for the targeted region of the intestinal tract, (c) producing the selected construct with an enterically coated packaging then (d) administering the packaged selected construct to a subject.
123. A method of delivering a monomeric polypeptide to the intestinal tract, comprising administering to a subject the construct or composition of any one of clauses 1 to 106.
124. The method of any one of clauses 116 to 119 or 122 to 123 wherein the subject is a mammal such as a human.
125. A method of preparing a product comprising a construct according to any one of clauses 1 to 104 which has been selected, the method comprising adding the selected construct into the product, wherein the selected construct is selected and produced by a method comprising the steps of: (a) performing the method according to either clause 120 or 121 then (b) selecting a construct with an appropriate level of lability for the targeted region of the intestinal tract.
126. A polypeptide comprising SEQ ID NO: 1, SEQ ID NO: 2, SEQ ID NO: 3, SEQ ID NO: 4, SEQ ID NO: 5, SEQ ID NO: 6.
127. A polynucleotide comprising SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36.

### Further Clauses

A set of further clauses defining the invention and its preferred aspects is as follows:
1. A construct suitable for oral administration comprising a first polypeptide and a second polypeptide connected by a labile peptide linker, wherein the labile peptide linker is labile to one or more proteases present in the intestinal tract and wherein the first and second polypeptides are substantially resistant to said one or more proteases.
2. The construct according to clause 1, wherein the first polypeptide is an antigen-binding polypeptide and the second polypeptide is an antigen-binding polypeptide.
3. The construct according to either clause 1 or 2 wherein the first and second polypeptides are substantially resistant to all proteases present in the intestinal tract.
4. The construct according to any one of clauses 1 to 3, wherein the labile peptide linker comprises at least 1, such as at least 2, such as at least 3, such as at least 4, such as at least 5, such as at least 6, such as at least 7, such as at least 8, such as at least 9, such as at least 10 K and/or R residues.
5. The construct according to any one of clauses 1 to 4, wherein all K or R residues comprised within the labile peptide linker have at least 1, such as at least 2, such as at least 3, such as at least 4, such as at least 5 shielding residues on their N-terminal and/or C-terminal side, wherein the shielding residues are selected from the list consisting of: D and E.
6. The construct according to any one of clauses 1 to 4, wherein all K or R residues comprised within the labile peptide linker have at least 1, such as at least 2, such as at least 3, such as at least 4, such as at least 5 non-shielding residues on their N-terminal and/or C-terminal side, wherein the non-shielding residues are selected from the list consisting of: C, A, S, N, G, L, I, V, T, M, F, Y, H, W and Q.
7. The construct according to clause 1, wherein the labile peptide linker comprises a polypeptide sequence of the format:

[—(GₐS)ₓ—U_{c}—B—U′_{d}—(G_{b}S)_{y}—]_{z}

wherein
   a is 1 to 10;
   b is 1 to 10;
   U is D or E;
   U′ is D or E;
   c is 0 to 7;
   d is 0 to 7;
   x is 1 to 10;
   y is 1 to 10
   z is 1 to 10 and
   B is K or R.
8. The construct according to clause 7, wherein c and d are both 0.
9. The construct according to clause 1, wherein the labile peptide linker comprises a polypeptide sequence of the format:

—B—(GₐS)ₓ—B′—

wherein
a is 1 to 10;
x is 1 to 10;
B is K or R and
B′ is K or R.
10. The construct according to clause 1, wherein the labile peptide linker comprises a polypeptide sequence of the format:

—B—(GₐS)ₓ—B′—(G_{b}S)_{y}—B″—

wherein
a is 1 to 10;
b is 1 to 10;
x is 1 to 10;
y is 1 to 10;
B is K or R;
B′ is K or R and
B″ is K or R.
11. The construct according to clause 1, wherein the labile peptide linker comprises a polypeptide sequence of the format:

[—(GₐS)ₓ—J—(G_{b}S)_{y}—]_{z}

wherein
   a is 1 to 10;
   b is 1 to 10;
   x is 1 to 10;
   y is 1 to 10
   z is 1 to 10 and
   J is W, F, Y, L or M; such as W, F or Y.
12. The construct according to any one of clauses 7 to 11, wherein a is 4, b is 4 and z is 1.
13. The construct according to any one of clauses 1 to 12, wherein the antigen-binding fragments are selected from the group consisting of: VLs, V-NARs, scFvs, Fab fragments, F(ab′)2 fragments or immunoglobulin chain variable domains such as VHHs and VHs.
14. A pharmaceutical composition comprising the construct according to any one of clauses 1 to 13 for use as a medicament for the treatment of diseases of the GIT administered by oral administration.
15. A polypeptide comprising SEQ ID NO: 1, SEQ ID NO: 2, SEQ ID NO: 3, SEQ ID NO: 4, SEQ ID NO: 5 or SEQ ID NO: 6.

The present invention will now be further described by means of the following non-limiting examples.

### EXAMPLES

### Example 1: The Trypsin Protease Assay

To test the lability of labile peptide linkers, the Trypsin Protease Assay was developed. This assay is performed as follows.

A buffered (10 mM acetic acid, pH 3.2, containing 0.01% thimerosal) aqueous suspension of L-1-tosylamido-2-phenylethyl chloromethyl ketone (TPCK)-treated Trypsin-agarose beads (trypsin from bovine pancreas; T4019; Sigma Aldrich) is used for the assay. The beads are washed 3 times with water (250 μl beads+1.25 ml water) followed by washing 5 times with Trypsin buffer (TRYP buffer; 1 mM Tris-HCl, 20 mM CaCl₂) [pH 8.0]). Finally, the resin is resuspended in TRYP buffer as a 50% (v/v) suspension.

100 μl of a 2 mg/ml construct solution is mixed with 225 μl 50% (v/v) immobilized TPCK-treated Trypsin in TRYP buffer. After time intervals such as 0, 10, 15, 30, 45 and 60 minutes of incubation at 37° C. in a shaker, samples are taken as follows: resin is pelleted by a 1 min centrifugation step at 500×g, and a 40 μl sample is taken from the supernatant and mixed with 2× sample loading buffer (such as Laemmli buffer). The remaining suspension is mixed again, and put back at 37° C. in the shaker.

For analysis, 15 μl of each sample is mixed with 5 μl 4× loading dye, boiled for 10 mins and 15 μl is loaded per lane on a polyacrylamide gel (such as NuPAGE 10% acrylamide Bis-Tris gel). Gels are run in SDS-MES buffer at 200 V for 35 mins. Gels are fixed in 40% methanol, 7% acetic acid for 30 mins and stained in colloidal Coomassie Brilliant Blue stain overnight. Gels are destained in water before imaging (such as using ImageQuant LAS4000 with 7 secs exposure). The quantity of intact constructs relative to cleaved constituent polypeptides can be assessed by comparing the corresponding bands in each time point lane.

### Example 2: The Chymotrypsin Protease Assay

The Trypsin Protease Assay protocol can be used wherein Trypsin beads are substituted with Chymotrypsin beads.

### Example 3: The Faecal Extract Protease Assay

To test the lability of labile peptide linkers, the Faecal Extract Protease Assay was developed. Faecal extract is a physiologically relevant matrix in particular for polypeptides targeted to the large intestine. This assay is performed as follows.

Human faecal samples from multiple individuals are turned into slurries with addition of 1×PBS at a ratio of 1, 2 or 3 mLs 1×PBS per gram of faeces. The slurries are then pooled (such that one pool represents the combined protease output from the faeces of multiple individuals), centrifuged and the supernatants removed, aliquoted and stored at −70 degrees C. This process removes the faecal matrix, including any cellular material. For digestion, constructs are incubated at a concentration of 160.16 μg/ml at 37° C. in pooled human faecal supernatant for 60 mins in the absence of BSA carrier. Aliquots are taken after time intervals such as 0, 10, 30 and 60 minutes and are mixed 1:1 in Protease Stop Solution (1×PBS+2× protease inhibitor cocktail (Sigma)+1% PMSF) and immediately frozen at −80° C.

For analysis, samples are defrosted and 15 μl of each sample is mixed with 5 μl 4× loading dye, boiled for 10 mins and 15 μl is loaded per lane on a polyacrylamide gel (such as NuPAGE 10% acrylamide Bis-Tris gel). Gels are run in SDS-MES buffer at 200 V for 35 mins. Gels are fixed in 40% methanol, 7% acetic acid for 30 mins and stained in colloidal Coomassie Brilliant Blue stain overnight. Gels were destained in water before imaging (such as using ImageQuant LAS4000 with 7 secs exposure). The quantity of intact constructs relative to cleaved constituent polypeptides can be assessed by comparing the corresponding higher and lower molecular weight bands in each time point lane.

### Example 4: Production of Constructs

Homobihead constructs containing a range of different linker sequences linking either (a) two anti-TcdA (Clostridium difficile toxin A) ICVDs were expressed in S. cerevisiae and (b) two anti-TNF-alpha ICVDs were designed and expressed in E. coli. The anti-TcdA constructs were homobiheads of ICVD ID1A, separated by different linkers. The anti-TNF-alpha constructs were homobiheads of ICVD IDSF, encoded by polynucleotides which also included Flag-6His tags.

### Anti-TcdA Constructs

Homobihead constructs ID3A, ID4A and ID25A to ID28A were expressed from S. cerevisiae. These constructs do not carry any protein tags at either terminus. ID25A to ID28A, containing the labile peptide linkers, were generated by overlap PCR using ID1A encoding DNA as a template and cloned into the Sac/HindIII sites of shuttle vector pUR4547 (BAC) capable of replication in E. coli and S. cerevisiae. Expression, and full secretion into the supernatant, from S. cerevisiae was achieved from the shuttle vector via galactose induction. Finally, the constructs were purified using Capto S cation exchange resin (GE) and dialysed into 1×PBS.

Expression in S. cerevisiae and subsequent storage was carried out according to the Yeast Expression Protocol as detailed above.

### Anti-TNF-Alpha Constructs

ID55F to ID60F were cloned directly into the SfiI/BsteII sites of vector pMEK222 (QVQ). ID55F to ID60F carry C-terminal Flag-6His tags. The resulting plasmids were transformed into E. coli BL21 DE3 (Novagen) for expression and transport to the periplasm. Following overnight growth in Autoinduction medium Terrific broth (Formedium), cells were pelleted, frozen at −80 degrees C. and re-suspended in 1/10 the original volume of 1×PBS to disrupt the outer membrane. ID55F to ID60F were purified from the supernatant by AKTA Prime FPLC using a Ni-NTA column to bind the His tags, followed by imidazole elution and dialysis into 1×PBS.

The sequences of the linkers are given in Table 1 below.

TABLE 1

| Con- | | | Linker | Construct |
|---|---|---|---|---|
| struct | ICVD | Linker | SEQ ID | SEQ ID |
| Name | Target | Sequence | Number | Number |
| ID3A | TcdA | GGGGSGGG | 1 | 7 |
| | | GSGGGGSG | | |
| | | GGGS | | |
| ID25A | TcdA | GGGGSKGG | 2 | 8 |
| | | GGS | | |
| ID26A | TcdA | GGGGSDKD | 3 | 9 |
| | | GGGGS | | |
| ID27A | TcdA | GGGGSDDD | 4 | 10 |
| | | DKGGGGS | | |
| ID28A | TcdA | KGGGGSGG | 5 | 11 |
| | | GGSK | | |
| ID4A | TcdA | RGGGGSRG | 6 | 38 |
| | | GGGSR | | |
| ID55F | Human | GGGGSGGG | 1 | 12 |
| | TNF- | GSGGGGSG | | |
| | alpha | GGGS | | |
| ID57F | Human | GGGGSKGG | 2 | 13 |
| | TNF- | GGS | | |
| | alpha | | | |
| ID58F | Human | GGGGSDKD | 3 | 14 |
| | TNF- | GGGGS | | |
| | alpha | | | |
| ID59F | Human | GGGGSDDD | 4 | 15 |
| | TNF- | DKGGGGS | | |
| | alpha | | | |
| ID60F | Human | KGGGGSGG | 5 | 16 |
| | TNF- | GGSK | | |
| | alpha | | | |
| ID56F | Human | RGGGGSRG | 6 | 17 |
| | TNF- | GGGSR | | |
| | alpha | | | |

### Example 5: Assaying the Lability of Anti-TcdA Constructs Using the Trypsin Protease Assay

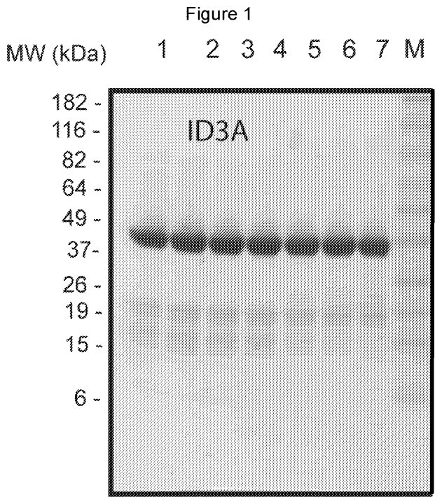

The lability of the constructs of Example 4 targeting TcdA were assayed using The Trypsin Protease Assay described in Example 1. The results of the assay are shown in SODS-PAGE gels in FIGS. 1 to 3 wherein M=molecular weight marker; lane 1=ICVD before addition of trypsin beads, lane 2=0 min digestion, lane 3=10 min digestion, lane 4=25 min digestion, lane 5=60 min digestion, lane 6=105 min digestion and lane 7=160 min digestion with trypsin beads.

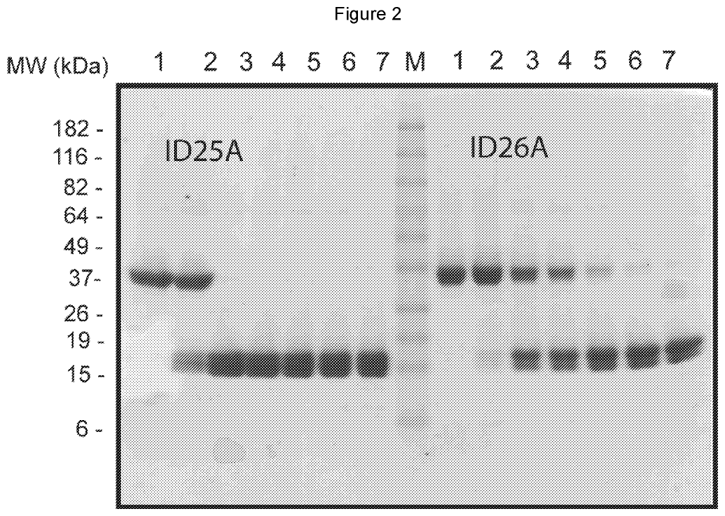

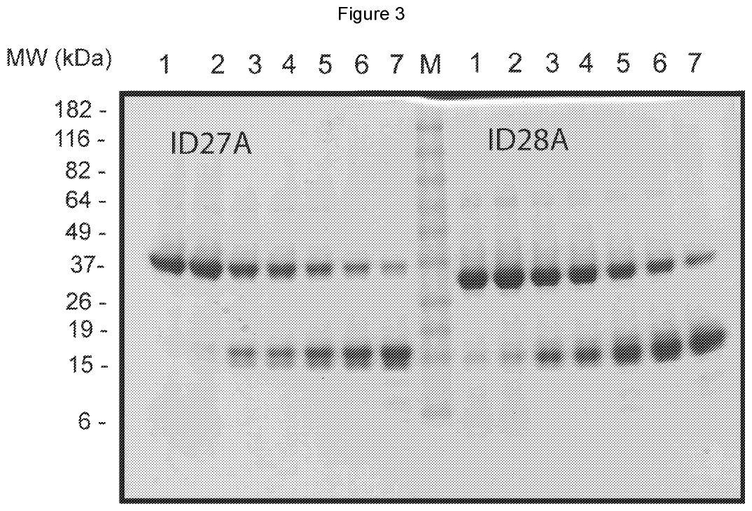

It can be seen that IM3A remained substantially intact over all time periods tested (FIG. 1). ID25A (FIG. 2) was completely cleaved very quickly after between 0 and 10 minutes of incubation. The shielding 0 residues in ID26A (FIG. 2) resulted in gradual cleavage of the linker from between 0 to 10 minutes to complete cleavage after 105 minutes. The labile residues situated at either terminus of the ID28A linker (FIG. 3) and the additional N-side shielding 0 residues in ID27A (FIG. 3) both resulted in slower cleavage, still ongoing after 160 minutes of incubation. The constructs in order of least to most labile were ID3A>ID28A>ID27A>ID26A>ID25A.

Uncleaved constructs running at molecular weights of approximately 35 kDa are visible in lanes corresponding to early time points (FIGS. 2 and 3). The cleaved constructs (i.e. constituent monomer ICVDs) are visible in lanes corresponding to later time points running at molecular weights of approximately 18 kDa. It is clear therefore from visual inspection of these gels that greater than 50% by mass of constructs ID25A, ID26A, ID27A and ID28A was cleaved into first and second immunoglobulin chain variable domains after 160 minutes (or earlier in some instances) after mixing in the Trypsin Protease Assay (lane 7). These are constructs of the invention comprising labile peptide linkers.

It is also clear from visual inspection of the gel in FIG. 1 that the only significant bands visible are those corresponding to the intact ID3A construct (running at a molecular weight of approximately 37 kDa). Therefore, ID3A was not cleaved into first and second immunoglobulin chain variable domains at any time point including after 160 minutes (lane 7) after mixing in the Trypsin Protease Assay. ID3A does not comprise a labile peptide linker and is not a construct according to the invention.

Furthermore, it is clear from visual inspection of the gels in FIGS. 2 and 3 that the first immunoglobulin chain variable domain and the second immunoglobulin chain variable domain in these constructs are themselves substantially resistant to one or more proteases present in the intestinal tract. It can be seen that there are no significant bands visible which correspond to fragments of ICVD monomers (the intact monomers running at a molecular weight of approximately 18 kDa) and thus at least 70% by mass of the immunoglobulin chain variable domains have clearly remained uncleaved after all time points tested including 160 minutes after mixing in the Trypsin Protease Assay.

### Example 6: Assaying the Lability of Anti-TcdA and Anti-TNF-Alpha Constructs Using the Faecal Protease Assay

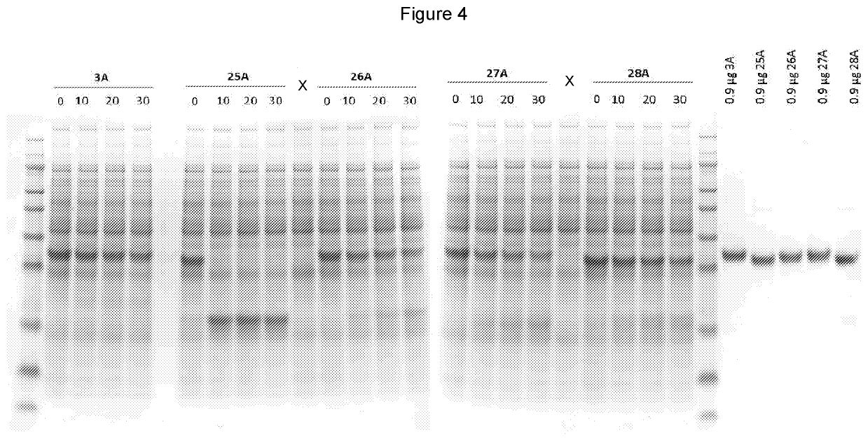

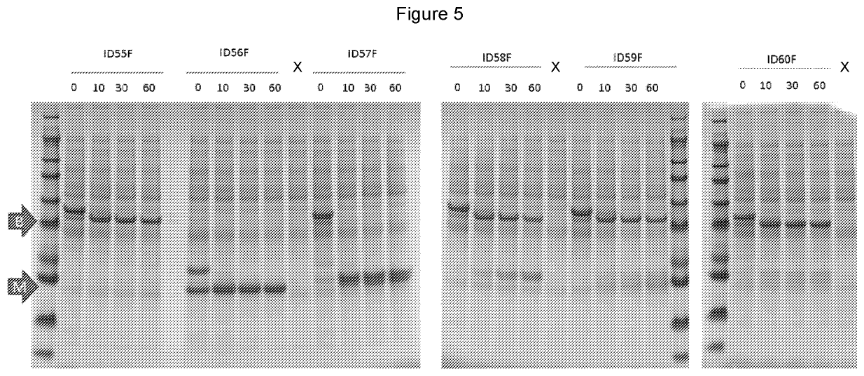

The lability of the constructs of Example 4 targeting TcdA and targeting TNF-alpha were assayed using The Faecal Protease Assay described in Example 3. The results of the assay are shown in SDS-PAGE gels in FIG. 4 (anti-TcdA constructs) and FIG. 5 (anti-TNF-alpha constructs). Lanes are labelled according to the construct tested and the period of incubation in minutes. ‘X’ indicates lane containing no ICVD (only faecal extract). In FIG. 5, ‘B’ indicates bihead and ‘M’ indicates cleaved monomers.

### Anti-TcdA Constructs

It can be seen from FIG. 4 that ID3A remained substantially intact over all time periods tested. ID25A was completely cleaved very quickly after between 0 and 10 minutes of incubation. The shielding D residues in ID26A, the additional N-side shielding D residues in ID27A and the labile residues situated at either terminus of the ID28A linker all resulted in slower cleavage relative to ID25A. Digestion of ID26A, ID27A and ID28A was still ongoing after 30 minutes of incubation.

### Anti-TNF-Alpha Constructs

It can be seen from FIG. 5 that ID55F remained substantially intact over all time periods tested. ID56F underwent instant total cleavage. ID57F was completely cleaved very quickly after between 0 and 10 minutes of incubation. The shielding D residues in ID58F, the labile residues situated at either terminus of the ID60F linker and the additional N-side shielding D restudies in ID59F all resulted in slower cleavage relative to ID57F. Digestion of ID58F, ID59F and ID60F was still ongoing after 60 minutes of incubation.

### Faecal Protease Assay Summary

It is apparent that lysine residues in the central region (but not peripheral regions) of the labile peptide linker increase lability to faecal proteases. Lability is reduced by shielding the lysine residues using flanking aspartate residues. The lability of the tested constructs in the Faecal Protease Assay is summarised in Tables 2 and 3 below.

TABLE 2

| Anti-TcdA | | | Linker |
|---|---|---|---|
| Construct | Linker | | SEQ ID |
| Name | Sequence | Lability | Number |
| ID3A | GGGGSGGG | Substantially | 1 |
| | GSGGGGSG | no cleavage | |
| | GGGS | | |
| ID28A | KGGGGSGG | Minor | 5 |
| | GGSK | cleavage | |
| ID26A | GGGGSDKD | Some | 3 |
| | GGGGS | cleavage | |
| ID27A | GGGGSDDD | Substantial | 4 |
| | DKGGGGS | cleavage | |
| ID25A | GGGGSKGG | Total | 2 |
| | GGS | cleavage | |

TABLE 3

| Anti-TNF- | | | |
|---|---|---|---|
| alpha | | | Linker |
| Construct | Linker | | SEQ ID |
| Name | Sequence | Lability | Number |
| ID55F | GGGGSGGG | Substantially | 1 |
| | GSGGGGSG | no cleavage | |
| | GGGS | | |
| ID59F | GGGGSDDD | Minimal | 4 |
| | DKGGGGS | cleavage | |
| ID60F | KGGGGSGG | Minor | 5 |
| | GGSK | cleavage | |
| ID58F | GGGGSDKD | Some | 3 |
| | GGGGS | cleavage | |
| ID57F | GGGGSKGG | Total | 2 |
| | GGS | cleavage | |
| ID56F | RGGGGSRG | Instant | 6 |
| | GGGSR | cleavage | |

### Example 7: Stability of Anti-TcdA Constructs During Storage

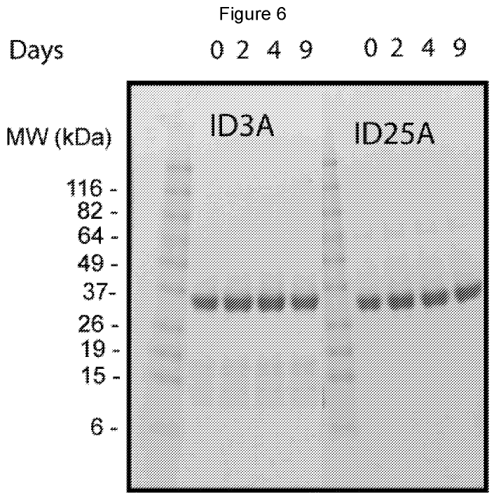

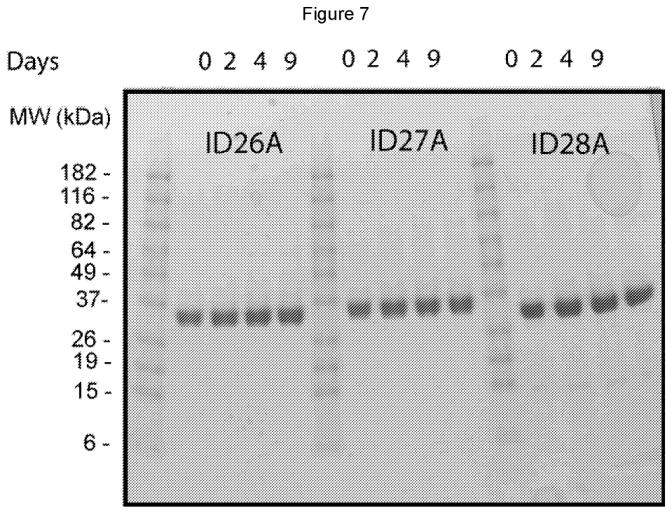

The anti-TcdA constructs (ID3A, ID25A, ID25A, ID27A and ID28A) were tested for their stability over time during storage. Samples were taken after 0, 2, 4 or 9 days of refrigeration at 4° C. (FIGS. 6 and 7). It can be seen that the SDS-PAGE gels contain bands corresponding to uncleaved bihead constructs and do not contain bands for cleaved monomers. Accordingly, the constructs are stable for at least 9 days under these conditions.

These uncleaved constructs ran with a molecular weight of approximately 35 kDa and constituent monomers would be expected to have an approximate molecular weight of 18 kDa. It is clear from visual inspection of the gels in FIGS. 6 and 7 that no significant bands are visible at a lower molecular weight than the 35 kDa bands corresponding to the intact constructs themselves and thus clearly no more than 10% by mass of these constructs is cleaved into first and second immunoglobulin chain variable domains after producing the constructs using the Yeast Expression Protocol (with 0 days storage) and also after a storage period of 2, 4 or 9 days.

Furthermore, it is clear that the constituent immunoglobulin chain variable domains are substantially resistant to yeast proteases because no significant bands running at a lower molecular weight than that of a single ICVD monomer (running at approximately 18 kDa) are visible in FIG. 6 or 7. Accordingly, it is clear that no more than 10% by mass of the first or second immunoglobulin chain variable domain is cleaved after producing the first or second immunoglobulin chain variable domain using the Yeast Expression Protocol (with 0 days storage) and also after a storage period of 2, 4 or 9 days.

### Example 8: Production of Constructs Incorporating Chymotrypsin-Labile Linkers

Homobihead or heterobihead constructs containing a range of different linker sequences containing chymotrypsin-labile sites (W, F, Y, L or M; particularly W, F or Y) may be designed and expressed in a suitable host such as S. cerevisiae, using the methods analogous to those detailed in Example 4 above. A suitable construct is provided in Table 4.

TABLE 4

| | | Linker |
|---|---|---|
| | | SEQ ID |
| ICVD Target | Linker Sequence | Number |
| TcdA | GGGGSYGGGGS | 37 |

The lability of such constructs is then assayed using the Chymotrypsin Protease Assay and the Faecal Protease Assay.

### Example 9: Testing the Stability of ID4A During Production and Storage

Anti-TcdA construct ID4A was expressed in S. cerevisiae using The Yeast Expression Protocol. ID4A is a homobihead of ICVD ID1A and does not carry any protein tag at either terminus. The sequence of the linker is given in Table 1 above.

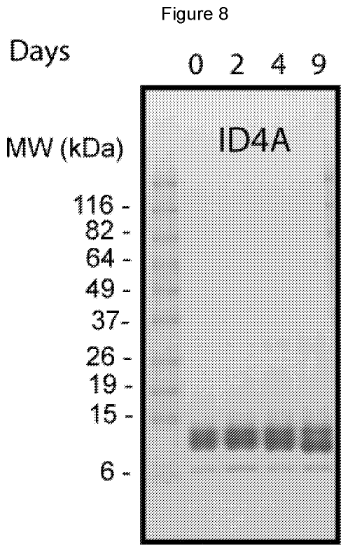

ID4A was tested for its stability over time during production and storage. Samples were taken after 0, 2, 4 or 9 days of refrigeration at 4° C. (FIG. 8). It can be seen that every lane of the SDS-PAGE gel contains bands corresponding to cleaved monomers, all running at an approximate molecular weight of 10 kDa and no lane clearly contains a band corresponding to uncleaved bihead. It appears that the linker used in ID4A was cleaved by yeast proteases during production.

It is therefore clear from visual inspection of the gel in FIG. 8 that more than 10% (in fact, 100%) by mass of ID4A is cleaved into first and second immunoglobulin chain variable domains after producing the construct using the Yeast Expression Protocol (with 0 days storage). Constructs of the invention are stable to yeast proteases and therefore ID4A is not a construct of the invention.

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps. All patents and patent applications mentioned throughout the specification of the present invention are herein incorporated in their entirety by reference. The invention embraces all combinations of preferred and more preferred groups and suitable and more suitable groups and embodiments of groups recited above.

1.-118. (canceled)

119. A labile peptide linker, wherein:
(a) the labile peptide linker comprises a polypeptide sequence of the format:
[-(GₐS)ₓ-B-(G_{b}S)_{y}-]_{z}
wherein
a is 1 to 10
b is 1 to 10
x is 1 or 10
y is 1 or 10
z is 1 to 10 and
B is K; and
(b) wherein the construct comprises a first VHH and a second VHH.

120. The labile peptide linker according claim 119, wherein the labile peptide linker is labile to one or more proteases present in the intestinal tract.

121. The labile peptide linker according claim 119, wherein the labile peptide linker is stable to yeast proteases.

122. The labile peptide linker according claim 119, wherein the labile peptide linker is labile to one or more proteases present in the intestinal tract such that greater than 50% by mass of the construct is cleaved into first and second VHHs after 160 minutes after mixing in the Trypsin Protease Assay.

123. The labile peptide linker according to claim 119, wherein the labile peptide linker is stable to yeast proteases such that no more than 10% by mass of the construct is cleaved into first and second VHHs after producing the construct using the Yeast Expression Protocol.

124. The labile peptide linker according to claim 119, wherein the first VHH and the second VHH are resistant to one or more proteases present in the intestinal tract such that at least 70% by mass of the first VHH and at least 70% by mass of the second VHH remain uncleaved after 10 minutes after mixing in the Trypsin Protease Assay.

125. The labile peptide linker according to claim 119, wherein the labile peptide linker comprises a cleavage site for trypsin or a trypsin-like protease.

126. The labile peptide linker according to claim 119, wherein the labile peptide linker has a length of at least 3 residues.

127. The labile peptide linker according to claim 119, wherein the labile peptide linker has a length of no greater than 40 residues.

128. The labile peptide linker according to claim 119, wherein the labile peptide linker comprises SEQ ID NO: 2.
